(19) United States
(12) Patent Application Publication
(10) Pub. No.: US 2009/0326677 A1
(43) Pub. Date: Dec. 31, 2009

(54) Joint Prosthetic Device

(75) Inventors: Sam L. Phillips, Largo, FL (US); Kathryn J. De Laurentis, Tampa, FL (US); Charles E. Pfeiffer, III, Phillipsburg, NJ (US)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: Fourier Designs, LLC, Largo, FL (US)

(21) Appl. No.: 12/491,648

(22) Filed: Jun. 25, 2009

Related U.S. Application Data
(60) Provisional application No. 61/075,557, filed on Jun. 25, 2008.

(51) Int. Cl. A61F 2/48

(52) U.S. Cl. 623/24; 623/57

(57) ABSTRACT

This invention provides a compact self-contained design of a wrist device for an electrically powered prosthesis. The wrist prosthesis, which provides user controlled motion in two directions, flexion/extension and rotation. Through the unique mounting of the one or more actuators or motors, a significant space savings is achieved. The modular terminal device distal mounting platform and prosthesis proximal mounting structure allow for this wrist device to function with many commercially available terminal devices and prosthesis designs. In a more particular embodiment related to a prosthesis the wrist device utilizes a system of gears, which achieves appropriately externally powered motion and is configured to provide wrist flexion as well as rotation. The prosthesis allows user controlled flexion/extension and rotation by providing two actuating motors and a series of gears, which provides externally powered flexion, extension, and rotation in various ranges.

This application claims the benefit of U.S. Provisional Application Ser. No. 61/075,557, filed Jun. 25, 2008, titled “Joint Prosthetic Device,” the entire contents of which are hereby incorporated by reference.

### FIELD OF THE INVENTION

Embodiments of this invention relate to an assistive device, such as prosthetic, orthotic, or other rehabilitative device, which improves the freedom of movement capabilities of people with artificial limbs. Specific embodiments of this invention are designed for the field of upper limb prosthetics, specifically multiple degree of freedom (DOF) (i.e., motion in two or more directions, or along two or more axes) wrist componentry. The present invention has two degrees of freedom that are externally powered by actuators such as motors or smart materials. Currently available wrist rotators and flexion devices have externally powered rotation only.

This invention also has utility anywhere an externally powered compact self-contained two DOF positioning device is needed where weight and power are a concern, such as wired or wireless still or video camera mounts; small unmanned exploratory machines; robotic arms; grasping devices; upper and lower limb rehabilitation devices; powered exercise equipment; bipedal/quadrapedal motion (i.e. leg movement) as an ankle joint; optical equipment; search and rescue systems; visual and retrieval inspection systems; directional systems to aim such things as antennae, lights, or solar panels; CNC machines; and any other appropriate applications.

When powered flexion/extension is added to rotation, it provides the user a much greater ability to orient the hand. This is particularly important in activities close to the midline, such as eating, dressing, or grooming. In engineering terms, the additional degree of freedom provides a six (DOF) system, which allows the hand to reach any point in the work envelope. Furthermore, the increase in the number of externally powered degrees of freedom that this wrist device provides aims to have a significant impact on the development of new methods of rehabilitation, creation of a dynamic research tool for scientists, and increased commercial applications beyond traditional prosthetics to such areas as robotics and telemedicine. Example applications are in wheelchair robotic arms and space robotic hands, since there also is a need for a compact self-contained mechanism that offers additional mobility and functionality for these grasping devices.

### BACKGROUND

It has been estimated that major amputations occur in one out of 300 individuals in the United States. There were 1,752,838 amputees in the US in the year 2000. That number is expected to increase to 2,382,413 amputees by 2020. There are 26,000 upper extremity amputations performed per year in the United States of which 10,000-12,000 would be likely candidates for embodiments of the wrist units described in this application.

Most upper extremity prostheses currently in use have a terminal device (hand or hook) controlled either by movements of the shoulder girdle transmitted via a cable (body powered), or by myoelectric control (motors are triggered by the contraction of muscles in the residual limb also referred to as electromyographic signals (EMG)). Myoelectric control was developed for commercial use beginning in the 1960s. A common complaint with myoelectric prostheses is the increased weight of the prosthesis, due to the motors powering the hand. Among the most difficult problems to be solved are multi-channel control of multiple degrees of freedom, and provision of sensory feedback.

The most commonly used myoelectric hand has one degree-of-freedom, opening and closing an anthropomorphically designed hand where the tip of the thumb contacts the tip of the second and third digits in a three-jaw chuck grip. It provides limited ability to grasp and manipulate small objects, while control of the force of the grip is very gross. Furthermore, the hand has a one degree-of-freedom passive wrist allowing only for rotation by the other arm unless a powered rotation wrist is added. That is, the person must use their healthy, non-prosthetic clad arm to rotate the wrist.

Improvement for upper limb prostheses is particularly important. Unlike lower limb prosthetics where biomechanics or weight activation control prosthetic function, the upper limb function cannot be controlled so easily. In other words, the user can activate a lower limb prosthetic device to move, sit, or stand via gravity or his/her own body weight during a natural gait or standing movements. An upper limb must be actively moved by an outside means such as a motor or cable wrapped around the healthy limb. Furthermore, gravity is a disadvantage.

The need for improved prosthetic devices is clear. Myoelectric prostheses users have reported rejection rates of up to 50% and only about 25% would rate themselves as excellent users. Additionally, 90% of those surveyed also had another type of prosthesis (cosmetic or body powered) that was preferred over the current myoelectric type. Richard Sherman studied traumatic amputees in the VA and found 22% said the prosthesis was not good for anything and only 32% reported the prosthesis was up to half as good as the original limb. Clearly, the current generation of powered upper limb prostheses is not serving the population as effectively as possible.

Furthermore, there has always been a fundamental problem between the development of improved control systems and adding more DOF to upper limb prostheses. The lack of control limits device development, and the lack of mechanical DOF limits improvements in control strategies.

Wrist flexion has been long recognized as important for upper extremity prosthetics for activities near the midline such as eating, toileting, shaving, and dressing. It is particularly important for the bilateral patient who must utilize the prosthesis for all activities of daily living. Powered flexion is an important addition to an externally powered prosthesis. Flexion and extension, along with rotation, give the prosthesis two additional degrees of freedom, which when added to the degrees of freedom for the shoulder and elbow yield a six-degree of freedom system. Six-degrees of freedom are important because they allow placement of the hand throughout the entire workspace (that is, the surrounding area in which one normally performs activities of daily living). Without flexion, amputees cannot reach a number of positions and must use compensatory motions for many other movements. These awkward motions are frequently cited as reasons for discontinuing prosthetic use.

Because the current state of the art allows the bilateral patient to reach certain positions in space by using compensatory motions only, the ability to flex the wrist is critical and may be an important link to reducing the rejection rate of powered prosthesis. In order to properly orient the terminal device, at least two degrees of freedom in the wrist unit are required. Previous attempts either have no ability to move or are passively moved in the flexion/extension plane. When both rotation and flexion have been powered, the resulting device has been too large, too heavy, or designed in such a manner to make it difficult to attach to a residual limb. Previous works to address this critical issue include Archer, U.S. Pat. No. 7,144,430, Jacobsen, U.S. Pat. No. 4,613,331, and May, U.S. Pat. No. 4,156,945, all which provide at least one degree of passive movement attained through manipulation by the opposite hand. Other designs, such as those from Pinson, U.S. Pat. No. 4,246,661, Maeda, U.S. Pat. No. 4,986,723, and Singleton, U.S. Pat. No. 6,817,641 are either too large or complicated to function effectively in the clinical environment. Rouse U.S. Pat. No. 7,048,768 provides a flexion and rotation unit, which is not externally powered and requires activating (body powered) movements, and there is no mechanism to connect power to the terminal device. This device, marketed by Texas Assistive Devices, demonstrates that a flexion/extension/rotation wrist is desired.

Other examples of flexion/extension/rotation technology are exemplified in motorized mounts for surveillance or hand held video cameras. These devices typically have motorized rotation (pan), but not all have motorized flexion and extension (tilt). They vary in range of motion, size, and lifting or torque capabilities, depending upon what type of camera they are designed for. Only those devices intended for use with hand held cameras are battery operated. These devices are significantly slower (6° per second, which is equivalent to 1 rpm) than what is required for a prosthetic device. One would expect this since a slow motion is required for capturing good pictures. Some examples are: the Pan & Tilt, from C. S. Lilin, the Pan Tilt Head from eBenk, and the Ninja Pan 'n Tilt from X10. In general, these are large and heavy, for example the Pan & Tilt has dimensions as follows: diameter=8 in (203 mm), length=11 in (279 mm), and weight=17 lbs (7.7 kg). These devices meet the needs of the camera industry in which they were designed; however, it is clear that they are not a viable choice for a device meant for powered flexion/extension and rotation with prosthetic hands or any for other small and lightweight uses. Their large size, slow movement, and often-small range of motion do not address the specific issues for a prosthesis.

### SUMMARY OF EMBODIMENTS OF THE INVENTION

Specific embodiments of the present invention seek to improve the mobility of upper limb prosthetic devices. There is provided an assistive device, which more accurately simulates the mobility of human limbs than currently available prostheses.

Embodiments provide an assistive device that has powered flexion.

Embodiments also provide an assistive device that has powered flexion/extension with powered rotation.

Certain embodiments further seek to improve the functional capabilities of assistive devices.

Specific embodiments are compatible with current myo-electric prosthetic hands. They may have two actuators, one for rotation and one for flexion/extension. Non-limiting examples of ranges of movement provided by these actuators enable the wrist to pronate/supinate about 360°, to extend through a range of about 0° to 90°, and to flex through a range of about 0° to 60°. Movement is proportional to sensor signal level and will be controlled by user generated inputs. User generated inputs are most commonly captured with EMG sensors, for which the target population will typically need at least two available EMG sites. This wrist may function for trans-radial, trans-humeral and shoulder disarticulation amputation levels. Other examples of user generated inputs include switches, touchpads, servo electric, linear transducers, or force sensing resistors.

Further embodiments also have utility anywhere an externally powered compact self-contained two DOF positioning device is needed where weight and power are a concern such as wired or wireless still or video camera mounts; small unmanned exploratory machines; robotic arms; grasping devices; upper and lower limb rehabilitation devices; powered exercise equipment; bipedal/quadrapedal motion (i.e. leg movement) as an ankle joint; optical equipment; search and rescue systems; visual and retrieval inspection systems; directional systems to aim such things as antennae, lights, or solar panels; CNC machines; and any other appropriate applications.

Specific embodiments of the invention provide a powered two degree of freedom wrist unit adapted for use with an artificial limb, the wrist unit comprising: a mounting platform adapted to connect to a modular terminal device; the mounting platform connected to an articulated member operated by an actuator system allowing for externally powered flexion and extension of the wrist unit; a rotation system associated with and operated by the actuator system allowing for externally powered rotation of the wrist unit; command circuitry associated with the actuator system for receiving user generated inputs; and a proximal mounting structure adapted to cooperate with and secure the wrist unit to a proximal prosthetic component.

In further embodiments, the user generated inputs comprise electromyographic signals, force resisting sensors, touchpads, linear transducers, pneumatic bladders, or switches.

The actuator system may comprise one or more actuators positioned to optimize rotational movement and flexion/extension movement. The actuators may be inverted, or are placed in parallel or perpendicular with each other.

There may be provided a lamination ring on the proximal mounting structure adapted to connect the wrist unit to a custom composite or thermoplastic socket. The proximal mounting structure is adapted to connect to a prosthetic elbow or bone anchored abutment. The proximal mounting structure may comprise an end portion of an outer housing or a rotation base.

The rotation system may comprise an outer housing and an actuator housing, the actuator housing adapted to be received by and rotate within the outer housing and to house the actuator system. Alternatively, the rotation system may comprise a rotation base and a wrist body, the wrist body adapted to be received by and rotate within the rotation base and to house the actuator system.

The wrist unit may provide increased mobility with ranges of at about 0-90° flexion, about 0-60° extension, and about 360° rotation. It may provide active lift of at about 1.5-10 ft-lbs and passive resist of about 10-30 ft-lbs. It may weigh about 0.5-1.00 pounds.

In some embodiments, the actuator system comprises power transmission devices such as springs, or AC motors, DC motors, solenoids, linear and rotary motors, or smart material advanced actuators that are battery operated.

The mounting platform, the articulated member, and the proximal mounting structure may comprise aluminum, plastic, carbon fiber composite, or composites or alloys thereof.

The command circuitry is typically adapted to be in electrical communication with user generated inputs, and wherein the command circuitry is further in electrical communication with the actuator system, such that when the user generated inputs are transmitted to the command circuitry, the actuator system responds by moving the wrist unit.

The wrist unit may be used in connection with a modular terminal device comprising a hand, a hook, or activity-specific device attached to the mounting platform. It may further be secured to a prosthetic component at the proximal mounting structure.

Further embodiments provide a powered prosthetic unit adapted to provide powered flexion, extension, and rotation to a modular artificial limb. The unit may be battery powered.

### BRIEF DESCRIPTION OF THE DRAWINGS

FIG. 1 shows an exploded view of one embodiment of a wrist device constructed in accordance with the present invention;

FIG. 1A shows an exploded view of one embodiment of a wrist device illustrating power transmission devices of FIG. 1 constructed in accordance with the present invention;

FIG. 1B shows an exploded view of one embodiment of a wrist device illustrating hidden componentry and structures of FIG. 1 constructed in accordance with the present invention;

FIG. 2 shows the assembled wrist device of FIG. 1;

FIG. 3 shows a side view of a wrist device of FIG. 1 according to one embodiment of the present invention illustrating placement of a gear assembly;

FIG. 4 shows a bottom view of a wrist device of FIG. 1 according to one embodiment of the present invention illustrating a gear assembly and inverted motor slot;

FIG. 5 shows an exploded view of a further embodiment of a wrist device constructed in accordance with the present invention;

FIG. 5A shows an exploded view of one embodiment of a wrist device illustrating power transmission devices of FIG. 5 constructed in accordance with the present invention;

FIGS. 6A and 6B show a wrist device of FIG. 5 according to one embodiment of the present invention demonstrating rotation.

FIG. 7 shows a wrist device of FIG. 5 according to one embodiment of the present invention demonstrating flexion.

FIG. 8 shows a wrist device of FIG. 5 according to one embodiment of the present invention demonstrating extension.

FIG. 9 shows an exploded view of one embodiment of a wrist device illustrating exemplary electronic and power components.

FIG. 10 shows an assembled wrist device of FIG. 9 illustrating electronic and power components attached to an actuator housing.

FIG. 11 shows an exploded view of a further embodiment of a wrist device constructed in accordance with embodiments of the present invention.

FIG. 12 shows an exploded view of a further embodiment of a wrist device.

FIG. 12A shows an exploded view of FIG. 12 excluding the motors, of one embodiment of a wrist device constructed in accordance with the present invention;

FIG. 13 shows an exploded view of a further embodiment of a wrist device.

FIG. 13A shows an exploded view of the wrist device of FIG. 13 illustrating power transmission devices.

FIG. 14 shows an assembled view of the wrist device of FIG. 13 illustrating hidden (internal) componentry.

FIG. 15 shows a bottom view of the wrist device of FIG. 13 illustrating power transmission devices and actuators.

FIGS. 16 to 38 are further images demonstrating various embodiments of the wrist devices described herein.

### DETAILED DESCRIPTION OF THE DRAWINGS

Reference will now be made to the exemplary embodiments illustrated in the drawings, and specific language will be used herein to describe the same. It should be understood that these descriptions and provided as examples only and are not intended to limit the scope of the invention. Alterations and further modifications of the inventive features described and illustrated, and additional applications of the principles described are considered within the scope of the invention.

Embodiments of the present invention relate generally to an innovative assistive device, specific embodiments of which are designed to improve the capabilities of artificial limbs using technologies developed in robotics. The assistive devices described herein are lightweight, compact and practical, which allows for more mobility than existing prosthetics. In certain embodiments, the assistive device relates to a modular externally powered wrist device. In specific embodiments, it relates to a modular externally powered two degree-of-freedom wrist device for primary inclusion in a prosthetic device that will complement the function of existing myoelectric hands, electric hooks, or activity specific terminal devices.

A specific feature of this invention relates to its externally powered flexion/extension and externally powered rotation. Powered flexion is an important addition to prosthetic systems because of the ability to achieve a greater workspace and work close to the midline. The increased ability to position the hand and work close to the midline of the body is important for many activities of daily living such as eating, dressing, and grooming.

A particular feature of this wrist includes a proximal mounting structure adapted to be releasably mounted to a patient, which allows the wrist to be integrated into a prosthesis by normal methods used by a prosthetist to fit a prosthesis to the user. The proximal mounting structure may include a lamination ring adapted to connect to a composite or thermoplastic socket, a prosthetic elbow, or a bone anchored abutment.

Another feature is a distal mounting platform, adapted to be connected to an articulated member that allows the wrist to be attached to a commercially available terminal device, such as an externally powered hand. The articulated member may be a support structure for movement of the distal device about the wrist device, such as a bracket, a flexion/extension bracket, a flexor, or an extender. The modular wrist system provided allows the user to determine which terminal device is best for him/her. If a wrist is integrated into the hand, then patients are restricted to only the one terminal device. Different modular terminal devices, such as hands, work hooks, and activity specific devices provide distinct functions.

Another feature of the design is its actuator system, which in a specific embodiment includes two actuated motions, rotation and flexion/extension. Power transmission devices or actuators, alone or in combination, may be used such as springs, AC motors, DC motors, solenoids, linear and rotary motors, smart material advanced actuators, or other electrically controlled power transmission devices. The number of actuators used may be one or more. The actuators may be assembled in the wrist device via a specialized configuration in which the motors are placed in parallel or perpendicular with each other, either inverted or not, or axially or perpendicular to the connecting members such as housings, plates, brackets or axles that join the power transmission devices, actuators, motion transmission devices, electronics, and control boards that make up the device.

The articulated member, which connects to the distal mounting platform for the purpose of flexion or extension of the terminal device, may be considered one of the connecting members described herein. The articulated members also connects to other housings and components, which may also be considered part of the connecting members described herein.)

One feature of motor torque requires an active lift equal to about 1.5-10 ft-lbs and passive resist of about 10-30 ft-lbs.

Another feature of the design is the compact size and low weight.

Low mechanical friction devices such as steel rods, rings, and bearings may be integrated into the joints as necessary to ensure durability, low friction, and quiet smooth motion. Gearing may be incorporated to ensure complete rotation of the hand in as short as time as possible, and in some instances about 1 second, or 1-2 seconds. These devices and the actuators are placed inside or along side connecting members or housings or attached to one another by such items as plates or brackets. Also included are connectors such as screws, nuts, bolts, threaded rods, rings, threads, pins, rivets, couplings, or other types of fasteners meant to join any connecting members mechanisms or transmission devices. The connecting members, mechanisms, connectors or transmission devices encompass lightweight materials such as aluminum, plastic, carbon fiber composite, composites, or any other appropriate material.

A further feature of this device is its use of a single battery to power both the hand and wrist for an appropriate length of time without recharging. In one embodiment, the single battery may last a minimum of ten hours of functional use without recharging. Another feature is sensory logic and peripheral circuitry constructed to fit inside the wrist for a compact single unit device.

The present invention may be used in applications other than in prosthetics as well. For example, there is provided relates to any an externally powered compact self-contained two DOF positioning device where weight and power are a concern such as wired or wireless still or video camera mounts; small unmanned exploratory machines; robotic arms; grasping devices; upper and lower limb rehabilitation devices; powered exercise equipment; bipedal/quadrapedal motion (i.e. leg movement) as an ankle joint; optical equipment; search and rescue systems; visual and retrieval inspection systems; directional systems to aim such things as antennae, lights, or solar panels; CNC machines; and any other appropriate applications.

Generally, embodiments of the invention provide assistive devices, such as prosthetic, orthotic, or other rehabilitative device, that improves the freedom of movement capabilities of people with artificial limbs. The wrist device shown and described is designed for upper limb prosthetics, specifically multiple DOF wrist componentry. It is designed to connect proximally to any commercially available prosthesis and distally to any commercially available terminal device. The compact wrist device provides many advantages for amputees because of the externally powered rotation and externally powered flexion/extension.

For example, in one embodiment, the wrist device includes a rotation system that comprises a cylindrical outer housing that is connected to an inner actuator housing, holding an actuator system comprising two motors, attached to an upper housing that includes an articulated member, such as a flexion/extension bracket. The actuator housing and upper housing rotate inside the outer housing when the rotation motor is powered. The flexion/extension bracket swings around and against the upper housing along a track in the upper housing when the flexion motor is powered.

Another example includes the flexion/extension bracket moving on top of the motor assembly where it is attached when the flexion motor is powered. In this example the motors are not encased in a housing but rather sandwiched between two plates; the top plate being where the flexion/extension bracket is attached. These examples include two motors, one to provide rotation and another to provide flexion/extension. Placed in specialized configurations, the rotation and flexion/extension motions can be achieved by one or many varied power transmission devices or actuators, alone or in combination, such as AC motors, DC motors, solenoids, linear and rotary motors, or smart material advanced actuators. Also included may be motion transmission devices such as bearings, bushings, or gears. Examples include a planetary gear assembly consisting of an inner gear, placed inside the outer housing with a spur gear attached to a motor to cause rotation; a bevel gear assembly placed inside the upper housing for the flexion and extension motions, where one bevel gear is attached to the second motor and the other is attached to a shaft that connects to the flexion/extension bracket to cause the bracket to rotate around the shaft effecting the flexion/extension motion.

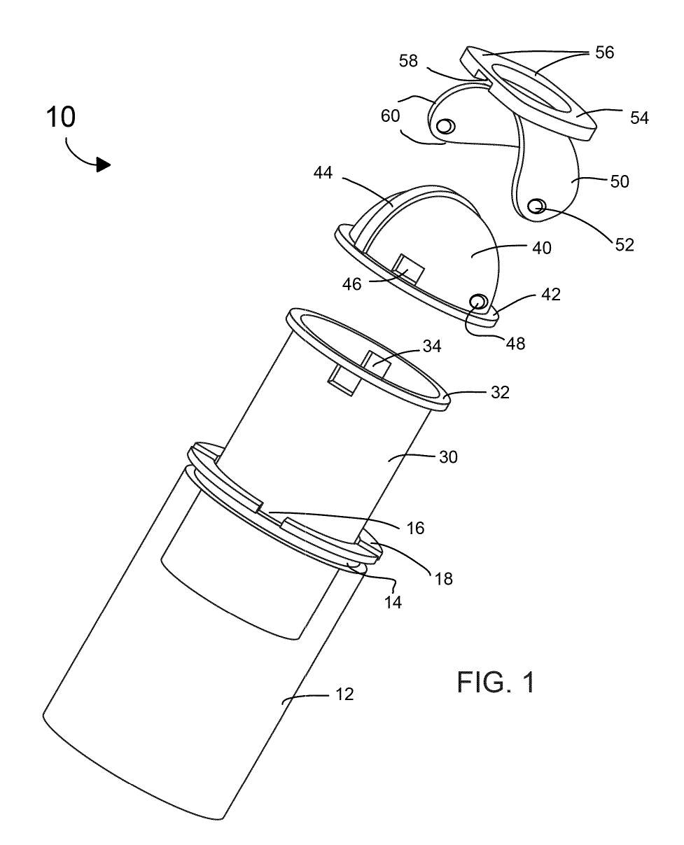

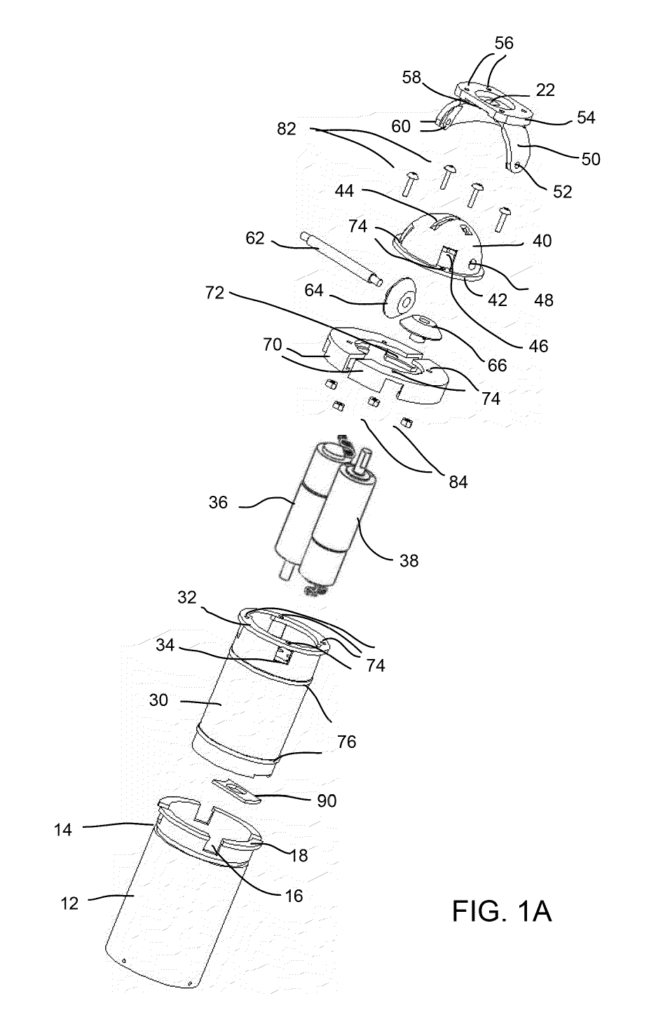

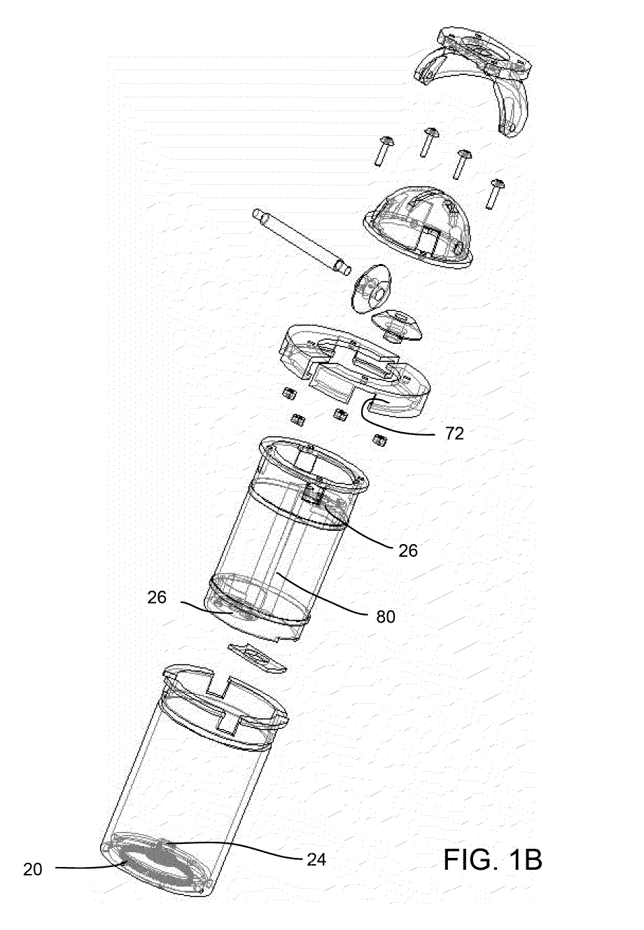

Referring now to FIG. 1, one embodiment of the wrist device 10 includes an outer housing 12 that is a hollow encasement to accept actuator housing 30 and inner gear 20, and provides the surface for attachment proximally to a prosthetic component. The outer housing 12 acts as a proximal mounting structure for the device 10. It also provides a portion of the rotation system, provided by cooperation between outer housing 12 and actuator housing 30. The proximal prosthetic mounting structure is adapted to attach to a composite or thermoplastic socket, prosthetic elbow, or bone anchored abutment.

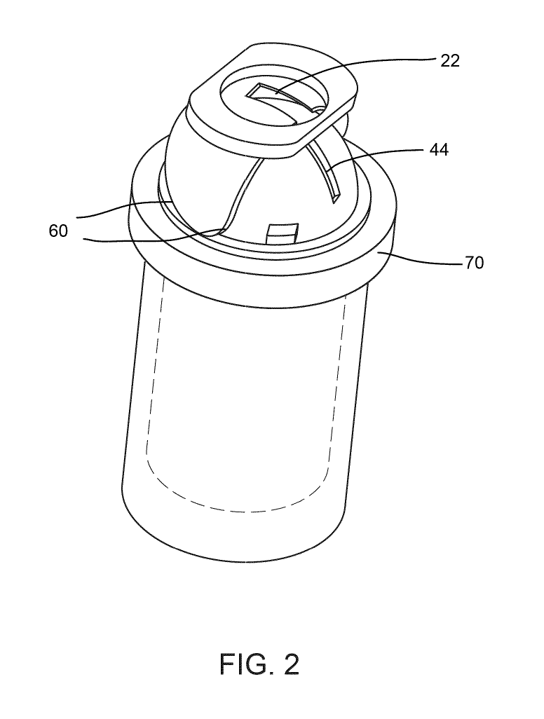

A circular hollow actuator housing 30 holds an actuator system, which in the shown example comprises the two motors 36, 38. Actuator housing 30 slides inside outer housing 12. An upper housing 40 sits on top of the outer housing 12 and actuator housing 30 and provides the base for an articulated member, which in the example shown is a wrist flexion/extension bracket 50. The upper housing 40 may be connected to outer housing 12 via connecting ring or rings 70, as shown in FIGS. 1A, 1B, and 2.

The outer housing 12 may be adapted to be attached to actuator housing 30 and upper housing 40 via screws 82, nuts 84, bolts, rings, threads, pins, rivets, couplings, or other types of fasteners. This attachment can be anywhere along outer housing 12, but is typically positioned near the top of outer housing 12. The outer housing 12 may be oval, cylindrical, circular, or conical in nature and is made of a lightweight material such as plastic, aluminum, alloy, composite, or any other appropriate material.

As shown in FIGS. 1 and 1A, outer housing 12 may have ribs or grooves such as a lamination ring 14, axially or circumferentially, on the outer surface to attach the prosthetic mounting structure, or it may be provided with any other appropriate attachment mechanism. The lamination ring 14 provides an area to fabricate the distal end of the custom socket attachment. The fibers of a custom composite prosthesis may be wrapped around lamination ring 14; then impregnated with resin creating a solid proximal wrist attachment. The lamination ring 14 may be used in transradial applications. Outer housing 12 is held stationary by the proximal connection such as a socket or prosthetic limb, which can be glued, cemented or otherwise attached to outer housing 12.

Actuator housing 30 includes a lip 32 at its top portion that has one or more screw holes 74 (and in a specific embodiment, four screw holes). Below the holes 74 are one or more slots 34 on actuator housing 30 (also slots 16 on outer housing 12 and slots 46 on upper housing 40). In a specific embodiment, the slots 34 are rectangular and the number of slots corresponds to the number of screw holes. The holes 74 are used for screws 82 to attach upper housing 40 and connector ring 70 to the top of outer housing 12. The slots 34 are to ensure access to screws 82. The lip 32 provides a surface for connecting ring 70 to fasten.

Motors 36 and 38 are attached to actuator housing 30 by brackets, plates, or cups 26 embedded or attached to the walls of actuator housing 30. In a specific embodiment, cups 26 are placed one facing toward the top and the other toward the bottom of actuator housing 30 with the openings facing each other for motor attachment. Each cup 26 has holes 78 for the shaft of the motor to pass through and screws to secure the motor to the cups 26. Cups 26, brackets, or plates are placed wherever necessary based on the dimensions of the motors. Cups 26, brackets, or plates may or may not be joined to each other with a connecting brace 80 (FIG. 1B). Cups 26, brackets, or plates are placed in such a way that the electronic cables can pass through the device. A device, motor cable bracket 90, may be used to keep actuator and electronic cables untangled and separate.

The actuator housing 30 may be a solid construction, or have slots or holes. The actuator housing 30 may be oval, cylindrical, circular, or conical in nature and is made of a lightweight material such as plastic, aluminum, alloy, composite, or any other appropriate material.

Actuator housing 30 attaches to upper housing 40 via screws 82, nuts 84, bolts, rings, threads, pins, rivets, couplings, or other types of fasteners and to outer housing 12 via screws 82, nuts 84, bolts, rings, threads, pins, rivets, couplings, or other types of fasteners. This attachment can be anywhere along the actuator housing 30, but is typically at its upper end.

The dome-shaped upper housing 40 holds wrist flexion/extension bracket 50 and attaches to the actuator housing 30. Upper housing 40 also has a rim 42 for attachment to the actuator housing 30. It may have a track or rib 44 running along its circumference, front to back, for the smooth consistent travel of flexion/extension bracket 50. The track or rib 44 of upper housing 40 allows for the smooth sliding of the flexion/extension bracket 50 over the upper housing 40 along path 22. Upper housing 40 can be spherical or partially spherical with slots or holes 46 or is open to allow access to the screws 82, bevel gears 64 and 66, and gear shaft 62. Upper housing 40 features holes 48 for the gear shaft 62 to slide through and to hold the flexion/extension bracket 50 through holes 52 for flexion/extension. The upper housing 40 has threaded holes for gear shaft 62 which may be partially or fully threaded with threaded inserts and nuts to keep the bevel gear 64 in proper alignment with bevel gear 66. Bevel gear 64 attached to gear shaft 62 connects to motor 38 with attached matching bevel gear 66. The upper housing 40 is made of a lightweight material such as plastic, aluminum, alloy, composite, or any other appropriate material.

The flexion/extension bracket 50 acts as an articulated member on which a mounting platform is allowed to articulate. Bracket 50 may be attached to the upper housing 40 via connection mechanisms such as threaded rods, rings, screws, nuts, bolts, threads, pins, rivets, couplings, or other types of fasteners. The flexion/extension bracket 50 may be partially spherical, symmetrical or nonsymmetrical. Its dimensions are intended to restrict the range of extension and flexion to desired values. The bracket 50 has holes 52 in it for the bevel gear shaft 62 attachment to bracket 50 via connection mechanisms such as threaded rods, rings, screws, nuts, bolts, threads, pins, rivets, couplings, or other types of fasteners. The gear shaft 62 goes through the upper housing 40 and the flexion/extension bracket 50 so that they are connected to each other. The flexion/extension bracket 50 is made of a lightweight material such as plastic, aluminum, alloy, composite, or any other appropriate material.

A mounting platform 56 is adapted to be attached or integrated to this bracket 50. Mounting platform 56 provides a mounting area for mounting and/or attachment of a terminal device. Mounting platform 56 may comprise a mount plate 54 having a mount plate holes 56 and slot 58. Mount plate 54 is made to fit an existing hand and copies the mount base of a terminal device. Mount plate's 54 outer surface may be modified to restrict the desired range of motion of the bracket 50 and may include slot 58.

In FIGS. 1 and 1A, the rounded edges 60 of the flexion/extension bracket 50 are an embodiment of the present invention that determines the specific angles for flexion (range of about 0°-90°) and extension (range of about 0°-60°). It should be understood that any appropriate ranges may be selected and designed for appropriate motion and other ranges are considered within the scope of this invention. The bracket 50 with rounded edges 60 around holes 52 also for smooth rotation movement about the upper housing 40.

Bracket 50 may also have a channel 22 that corresponds with the shape of track 44 for smooth movement of the flexion/extension bracket 50 about the upper housing 40. The outer dimensions of mount plate 54 fit the flexion/extension bracket 50 so that the bracket 50 moves within the about 0°-90° range or any other appropriate selected range. Mount plate 54 has mount plate holes 56, which placement replicates that of the mounting plate of an existing prosthetic hand.

As shown in FIGS. 1A, 1B and 2, ring 70 connects the device 10 together. Ring 70 is attached to actuator housing 30 and upper housing 40 with the attached flexion/extension bracket 50. It is attached via connection mechanisms such as screws 82, nuts 84, bolts, threaded rods, rings, threads, pins, rivets, couplings, or other types of fasteners. It can be on the outside or inside of the housings and may be attached with or without threads. The ring 70 may fit in multiple pieces or a series of rings on the outside or inside of the outer housing 12. In one embodiment, the ring 70 may be split into two pieces attached to the outside of outer housing 12 as in FIGS. 1A and 1B. It may have holes or slots 74 for attachment between the actuator 30 and upper housing 40. The inside of ring 70 may have a U-shaped channel 72 so that it hooks under the lip 18 on the outer housing 12 so that the actuator housing 30, upper housing 40, and flexion/extension bracket 50 rotate together inside the outer housing 12. This provides a rotation system. Connecting ring 70 is adapted to be attached around lip 18 (outer housing 12), lip 32 (actuator housing 30), and rim 42 (upper housing 40) via U-shaped channel 72. In a specific embodiment connecting ring 70 joins outer housing 12, actuator housing 30, and upper housing 40. Ring 70 is made of a lightweight material such as plastic, aluminum, alloy, composite, or any other appropriate material.

One benefit of device 10 is that it provides smooth adjacent movement of attached outer housing 12, actuator housing 30, and flexion/extension bracket 50. This is achieved via transmission devices such as bearings, bushings, flanges, or rings. The inside surface of outer housing 12 may be smooth or may have bearings, bushings, flanges, or rings embedded or attached, axially or circumferentially, to allow smooth travel during rotation. The outside surface of actuator housing 30 may be smooth or may have bearings, bushings, flanges, or rings embedded or attached, axially or circumferentially, to allow smooth travel during rotation. It generally has a smooth surface inside and out. The mechanisms for smooth rotation may be placed inside the outer housing 12 or on the outside of the actuator housing 30 such as the sleeve bearing ribs 76. Gear shaft 62 may have bearings, bushings, for smooth travel of flexion/extension bracket 50 during flexion and extension.

The rotation and flexion/extension can occur via a variety of power transmission methods such as gears, grooves, cams, belts, sprockets and pulleys, or mechanisms along with actuators. The gears, gear assembly, gear assemblies, or mechanisms for rotation may be placed in the outer housing 12 or on the inside of actuator housing 30 or on the inside of upper housing 40. The gears, gear assembly, gear assemblies, or mechanisms for flexion/extension can be placed anywhere within outer housing 12 or inside of actuator housing 30 or inside of upper housing 40. The gears can consist of two or more gears such as worms/worm gears, planetary, bevel, miter, helical, spur, rack and pinion, or Geneva, or any other appropriate gear or mechanism. The gear ratio of said gearing mechanism introduced into the device is configured for the user depending upon desired torque/speed. The actuation, rotation and flexion/extension, may be from actuators or power transmission devices such as AC motors, DC motors, solenoids, linear and rotary motors, or smart material advanced actuators.

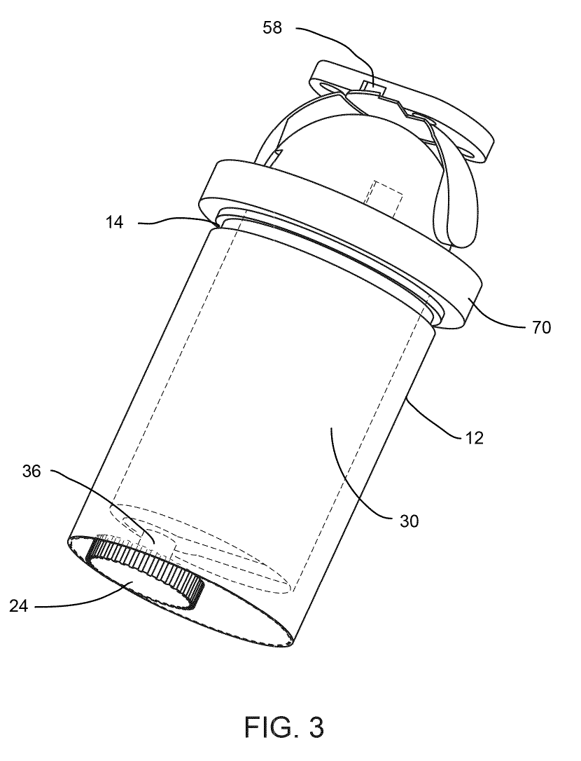

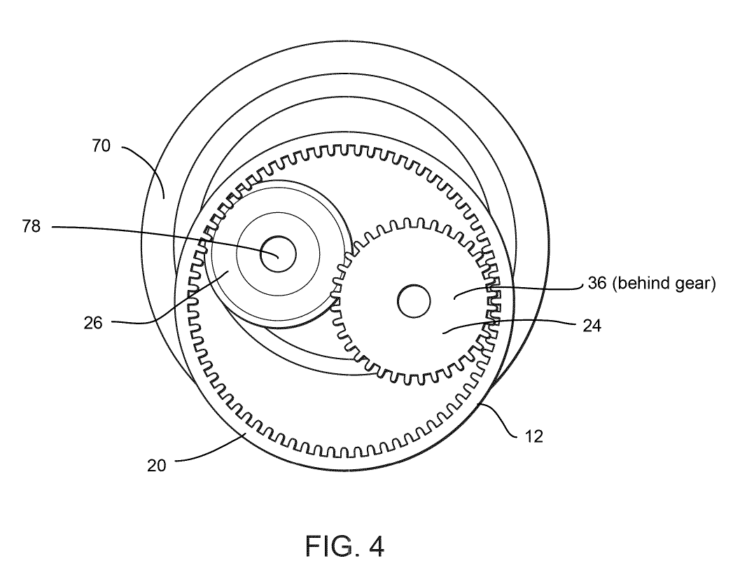

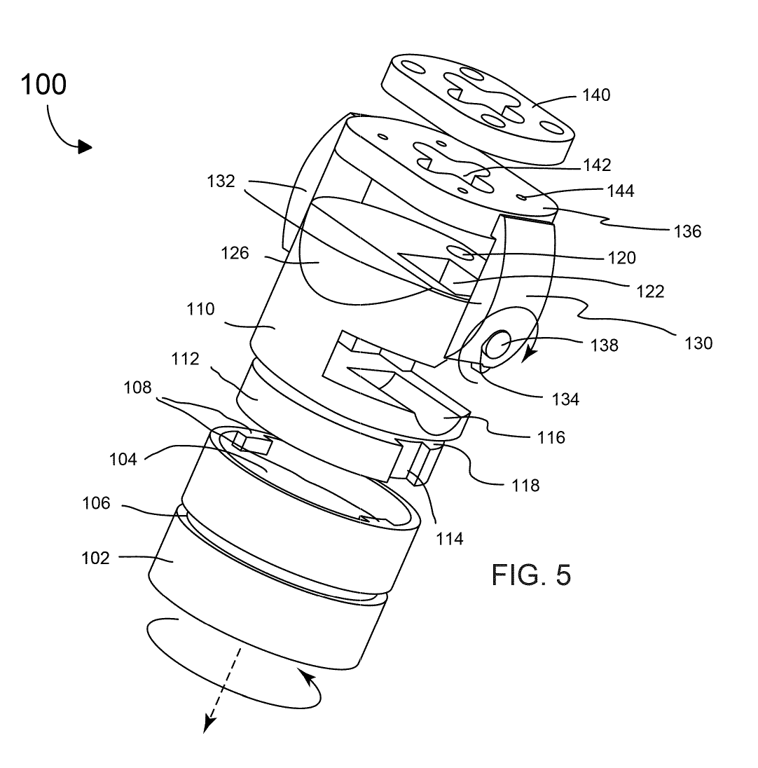

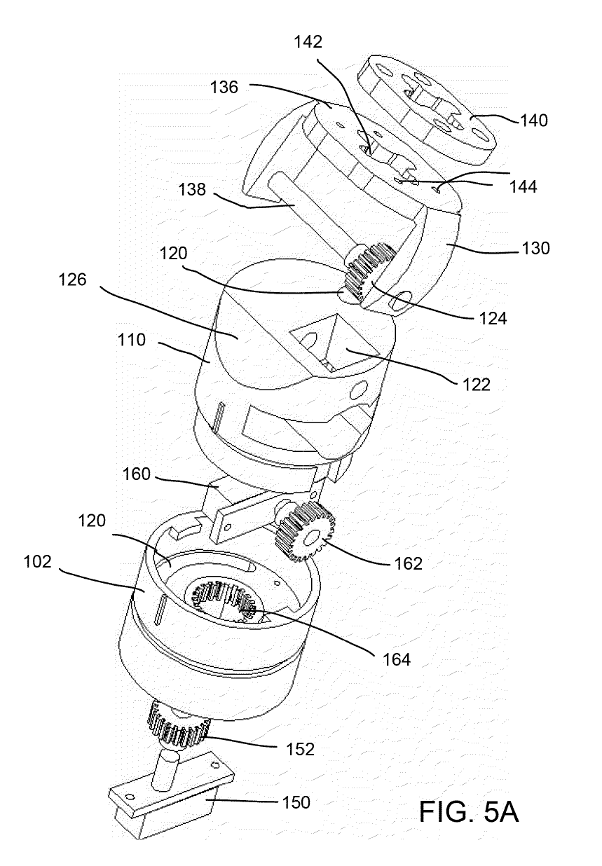

Actuator housing 30 holds one or more power transmission devices placed generally parallel to each other, inverted and axial to the actuator housing 30 as illustrated in FIG. 1A (or perpendicular to each other as illustrated later in FIG. 5A) or some other specialized configuration to provide rotation and flexion/extension of the device. For example, if the wrist is actuated via motors, the motor shafts can be facing perpendicular or parallel to one another, in the same or opposite direction. FIGS. 1A, 1B, 3 and 4 show an embodiment of the present invention that includes motors 36 and 38 placed parallel with each other in opposite directions.

FIGS. 1A, 1B, 3 and 4 show a view of the gear assembly consisting of inner gear 20 and spur gear 24 in which the inner gear 20 is adapted to be attached to or be a part of the outer housing 12. The gear assembly may be placed at the bottom or proximal end of outer housing 12 and actuator housing 30. A spur gear 24 that makes contact with an inner gear 20 is attached to one motor 36 for rotation. In a specific embodiment motor 36 provides the wrist device rotation of about 360°. The gear assembly consisting of bevel gears 64 and 66 may be may be placed at the top or distal end of the outer housing 12 and actuator housing 30, inside upper housing 40. A bevel gear 66 that makes contact with a bevel gear 64 is attached to one motor 38 for flexion and extension. In a specific embodiment motor 38 provides the wrist device flexion (range of about 0°-90°) and extension (range of about 0°-60°). Motors 36 and 38 produce active lift of about 1.5-10 ft-lbs and passive resist of about 10-30 ft-lbs.

The wrist device 10 is compact in size and low in weight. A specific embodiment has dimensions of a diameter of about 1.75-2.5 inches (44.5-63.5 mm), length of about 0.5-2.0 inches (12.7-50.8 mm) exposed between the hand and laminate, and about 1.0-3.0 inches (25.4-76.2 mm) in overall length. It weighs 0.5-1.00 lbs (242-484 g).

FIG. 5 shows an alternate embodiment of a wrist device. As illustrated in FIGS. 5 and 5A, the wrist device 100 includes a rotation base 102 that fits into the wrist body 110. Together these components provide a rotation system. The top portion, flexion/extension bracket 130, including mount base 136, provides the flexion/extension of the wrist. Attached to this is mount base plate 140.

The rotation base 102 is oval, cylindrical, circular, or conical in nature. Rotation base 102 may have ribs or grooves 106, axially or circumferentially, on the outer surface to attach the device 100 to a proximal prosthetic component, and as such, it acts as a proximal mounting structure. The inside surface is smooth or has ball bearings, flanges, or rings embedded or attached, axially or circumferentially, to allow smooth travel during rotation. Rotation base 102 is held stationary by a socket in the proximal prosthetic, which can be glued, cemented or otherwise attached the rotation base 102. The gears or mechanisms for rotation are placed in the rotation base 102. The rotation base 102 may also attach to the wrist body 110 via rings, screws, nuts, bolts, threads, pins, rivets, couplings, or other types of fasteners. Cooperation between the rotation body 102 and the wrist body 110 provides a rotation system that allows rotation of the device 100.

In FIGS. 5 and 5A, attachment ring 112 of wrist body 110 may fit inside rotation base 102 sliding past overhangs 108 in the rotation base 102 via notches 114 in the wrist body 110 such that the overhangs 108 to fit into channel 118 on the wrist body 110. Channel 118 is around the circumference of wrist body 110 and provides an alleyway for wrist body 110 to rotate within rotation base 102. One rotation motor 150 is housed inside the rotation base 102 for rotation. A spur gear 152 on rotation motor 150 fits inside an inner gear 164 attached to or embedded in wrist body 110. Rotation motor 150 can be placed axially with the base 102 or perpendicular to it. Wrist body 110 may be made of a lightweight material such as plastic, aluminum, alloy, composite, or any other appropriate material.

A cylindrical cutout 104 houses the rotation motor 150 axial to rotation base 102. One spur gear 152 is attached to rotation motor 150 and fits into an inner gear 164 inside the bottom of wrist body 110. Rotation base 102 has a slot 106 around its circumference for a socket or prosthetic limb attachment. Rotation base 102 houses the batteries and electronic control boards.

There may be holes 120 throughout wrist body 110 and rotation base 102 for cables to pass through wrist body 110 and rotation base 102. Wrist body 110 has cuts 126 on the outside to allow for flexion/extension bracket 130 to flex/extend to the desired range. Cut 126 may be redefined as necessary for desired angle range of flexion and extension.

In FIGS. 5 and 5A, an articulated member, which is shown as flexion/extension bracket 130 may be partially spherical, symmetrical or nonsymmetrical. Its dimensions are intended to restrict the range of extension and flexion to the desired values. The mount base pattern (mount plate holes 142 and 144) for a prosthetic hand is either attached or integrated to flexion/extension bracket 130. Mounting platform or mount plate 136 is made to fit an existing hand and copies mount base plate 140 of said prosthetic hand. The mount plate 136 outer surface may be modified to keep the desired range of motion of the flexion/extension bracket 130. Flexion/extension bracket 130 may be rounded for smooth rotation movement about the wrist body 110. Flexion/extension bracket 130 has holes 134 for the bevel gear shaft 138 attachment, which is attached to the flexion/extension bracket 130 via connection mechanisms such as threaded rods, rings, screws, nuts, bolts, threads, pins, rivets, couplings, or other types of fasteners. Gear shaft 138 goes through wrist body 110 and flexion/extension bracket 130 to connect each other. The flexion/extension bracket 130 may be made of a lightweight material such as plastic, aluminum, alloy, composite or any other appropriate material.

Mount plate 136 may have two arms 132 attached that have a hole 134 for gear shaft 138. Mount plate 136 is a certain specified distance from the top of wrist body 110 so that it rotates within about a 30°-60° range about the wrist body 110, which translates to about a 30°-60° range of flexion/extension, although it should be understood that other ranges are possible and within the scope of this invention. The gear shaft 138 slides through the holes 134 and is attached to the arms 132 via connection mechanisms such as threaded rods, rings, screws, nuts, bolts, threads, pins, rivets, couplings, or other types of fasteners.

The mounting platform or mount plate 136 is a replica of a mount base plate 140 for any prosthetic hand attachment that may be connected to the wrist device. The outer dimensions of the mount base 136 fit the flexion/extension bracket 130 so that flexion/extension bracket 130 moves within about 0°-60° range. For example, mount base holes 142 and 144 placement copies that of mount base plate 140 of an existing prosthetic hand.

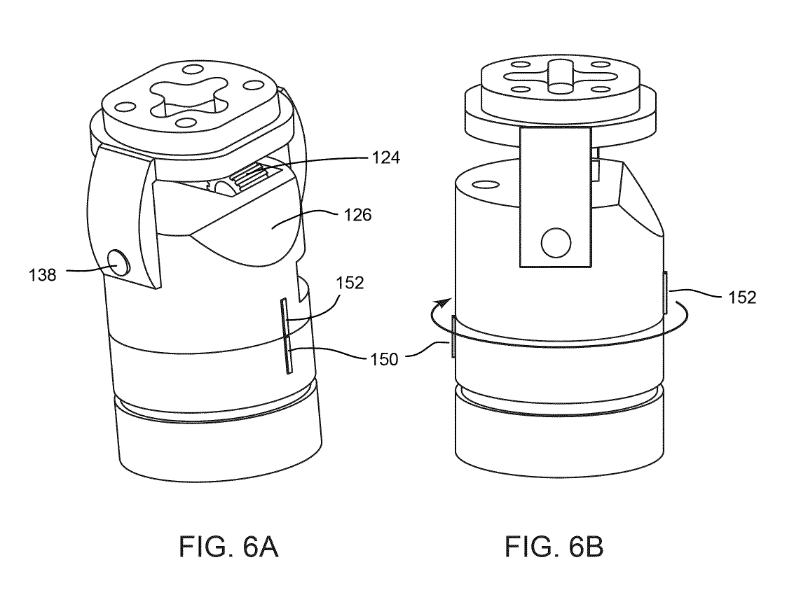

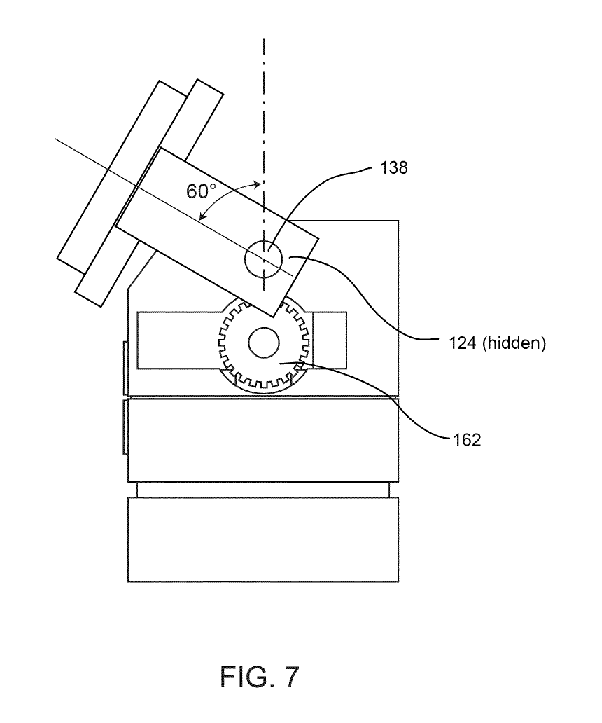

In FIGS. 5A and 6A, spur gear 124 attached to gear shaft 138 of flexion/extension bracket 130 matches up with spur gear 162 on flexion/extension motor 160. The flexion/extension motor 160 can be placed axially or perpendicular as it is illustrated here; motor 160 is placed perpendicular with the wrist body 110. As shown in FIG. 7, flexion/extension motor 160 which causes flexion has a spur gear 162 attached that matches up, through the hole or slot 122, with the spur gear 124.

FIGS. 6A and 6B show examples of the wrist device rotation. In one embodiment, rotation base 102 includes a bar 150 that lines up with a corresponding bar 152 on wrist body 110. These bars align when the wrist is not rotated. The alignment or misalignment of these bars 150 and 152 provide the means to mark the exact angle of rotation.

FIGS. 7 and 8 show examples of the wrist flexion/extension about gear shaft 138.

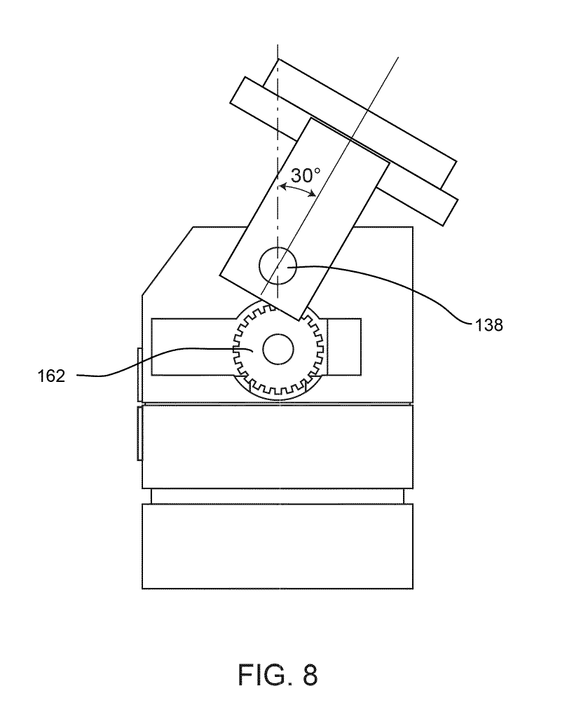

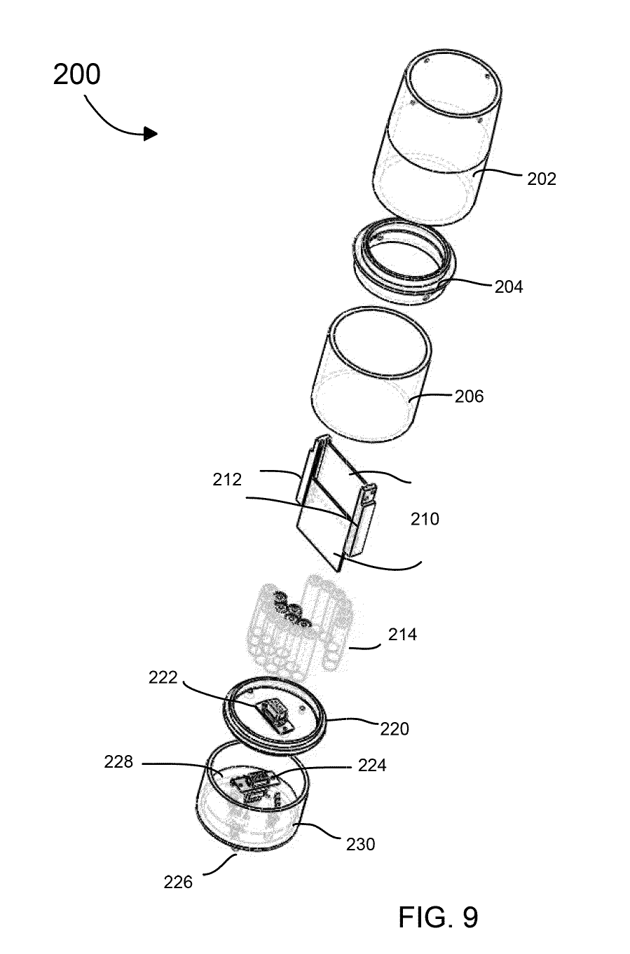

FIG. 9 shows a further embodiment of a wrist device. Wrist device 200 is designed to compactly and portably provide the power and control electronics for wrist devices 10, 300, and 400. Wrist device 200 is comprised of all electronics components to operate the wrist devices described herein for which the electronics components are non-limiting examples of the command circuitry that may be used to effect wrist movement. Specific embodiments of the present invention include wrist interface housing 202, battery and control electronics compartment 206, control boards 210, batteries 214, and interface control box 230.

The command circuitry may be associated with an actuator system (including motors) in order to be responsive to user generated inputs, which may include but are not limited to electromyographic signals, force resisting sensors, touchpads, linear transducers, pneumatic bladders, or switches.

Wrist interface housing 202 is designed to receive and is configured to be connected to power transmission devices or an actuator housing. Wrist interface housing 202 is connected to battery and control electronics compartment 206 via connector ring 204. Battery and control electronics compartment 206 is designed to receive and hold batteries 214, control boards 210, and control board brackets 212. Control boards 210 provide the circuitry to control the wrist and any prosthetic hand that may be attached to the wrist device. Control board brackets 212 hold control boards 210 and are adapted to be attached to the inside of battery and control electronics compartment 206. Batteries 214 supply power to the actuators of the wrist device and any prosthetic hand that may be attached to the wrist device. Specific embodiments of the present invention show individual batteries 214, which may be substituted with one comprehensive battery that performs the same functions as batteries 214. Batteries 214 may surround control boards 210 and control board brackets 212 for a compact wrist device design.

Battery and control electronics compartment 206 is configured to be connected to interface control box 230 via ring 220. Electronics connector 222 fits into interface connector 224, which is secured onto interface board 228. Electronics connector 222 can also be attached directly to command circuitry of any prosthetic hand bypassing interface control box 230, which is optional. Specific embodiments of the present invention show the interface control box 230 as the user interface to operate the wrist device for testing or demonstration. Interface connector 224 secured onto interface board 228, which is configured to be connected to switches 226, which operate the on/off control of the wrist device power transmission devices. Specific embodiments of the present invention show switches 226 numbering in three to operate three motors for wrist rotation, wrist flexion/extension, and hand opening and closing.

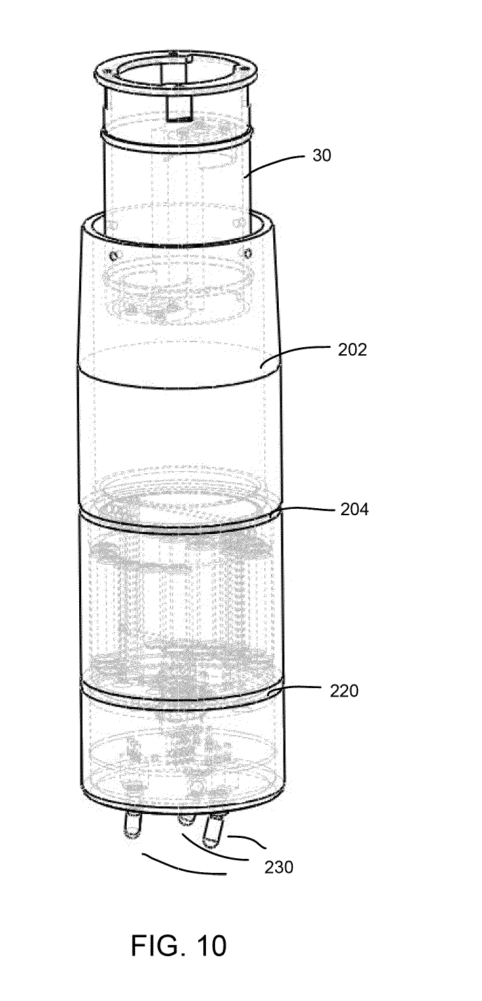

In FIG. 10, the wrist device 200 is assembled. Wrist interface housing 202 is adapted to attach to power transmission devices or an actuator housing as shown herein receiving actuator housing 30 (see FIGS. 1 and 1A).

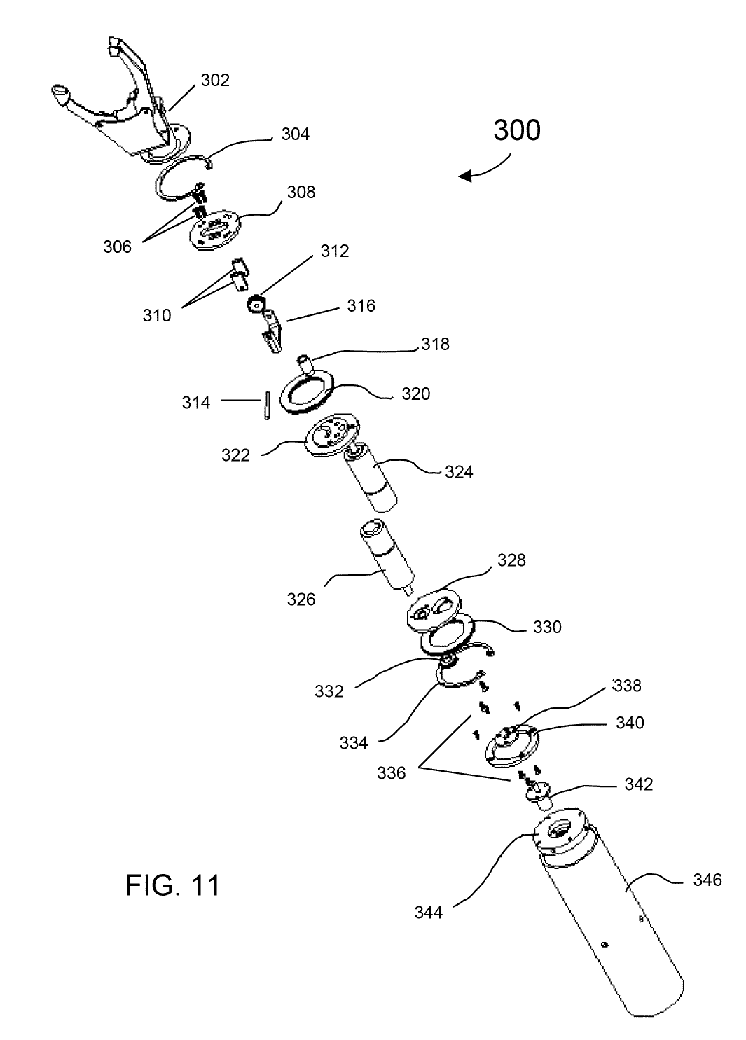

FIG. 11 shows a further embodiment of a wrist device. Wrist device 300 is designed to perform the same functions as wrist devices 10, 100, and 200. Wrist device 300 includes an outer sleeve 344 that is hollow. Outer sleeve 344 is configured to be connected to a proximal prosthetic component and acts as a proximal mounting structure. A mounting platform or an adapter plate 308 is customized for any hand attachment, which is attached to the bracket legs 310. Included are thrust bearing components 320 and 330 for smooth rotation of the wrist inside the outer sleeve and components. Elements 304, 306, 314, 334, 336, 338, 342 (the cooperation between each of which is described in further detail below) secure outer sleeve 346, internal gear 340, slip ring 344, bottom motor plate 328, top motor plate 322, wrist base bracket 316, and adapter plate 308 and bracket legs 310 to one another. The prosthetic hand 302 demonstrates any terminal device (not specifically considered part of this invention) that can be attached to the wrist device.

Specifically, a retaining clip 304 holds together the motor assembly (322, 324, 326, 328). Screws 306 are meant to hold adapter plate 308 to bracket legs 310, which are secured by wrist pin 314. The adapter plate 308 is custom made for a desired hand to be attached. The wrist pin 314 secures worm gear 312 and bracket legs 310 together to create motion. The worm gear 312 is for wrist flexion and extension. The wrist base bracket 316 attaches the motor assembly to the wrist/hand. The worm 318 is attached to motor 324 to drive the worm gear 312. The thrust bearing 320 is included to allow smooth moving contact between the retaining clip 304 and top motor plate 322. The top motor plate 322 anchors the top of the wrist motor 324 into position and retains the bottom of the pivot motor 326. The wrist motor 324 articulates the wrist motion (flexion/extension). The pivot motor 326 articulates the wrist rotation. The bottom motor plate 328 anchors the top of pivot motor 326 and retains the bottom of wrist motor 324. The thrust bearing 330 allows smooth moving contact between the bottom motor plate 328 and the retaining clip 334. The spur gear 332 attaches to the pivot motor. The retaining clip 334 holds the bottom of the motor assembly (322, 324, 326, 328). The screws 336 attach internal gear 340 to slip ring bracket 338. The slip ring collar 342 secures slip ring 344 into slip ring bracket 338. Internal ring gear 340 is attached to slip ring bracket 338, which is then secured to outer sleeve 346. Slip ring 344 passes all electrical conductors through a rotating mechanism without loss of conduction. Slip ring bracket 338 holds slip ring 344 and internal ring gear 340 in place and anchors to outer sleeve 346. Outer sleeve 346 holds the power and electronics in the bottom half and interfaces to an existing or new prosthetic arm.

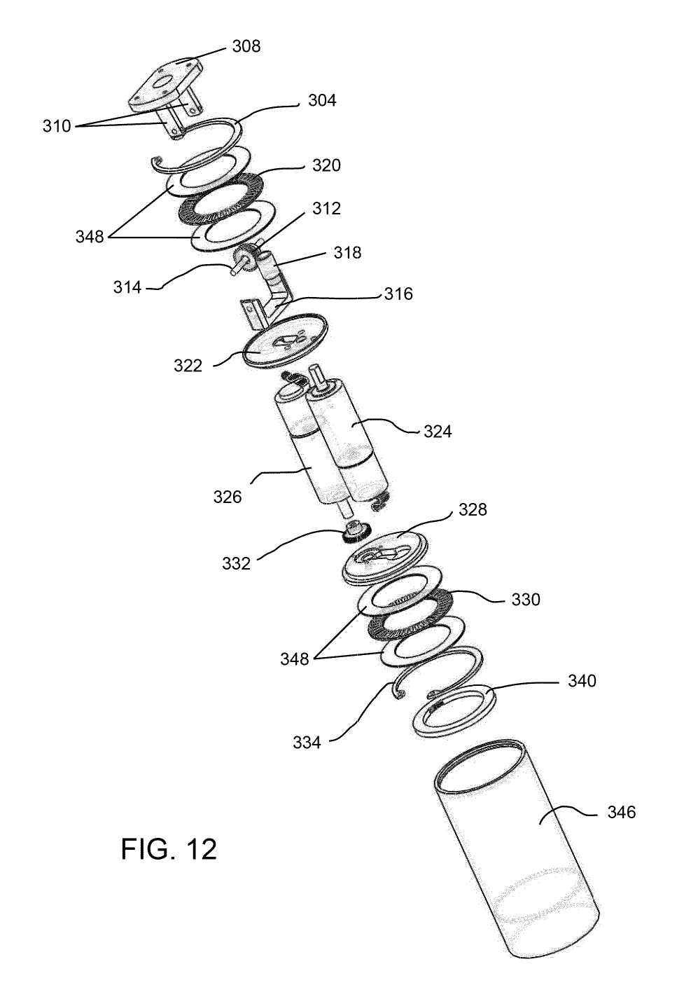

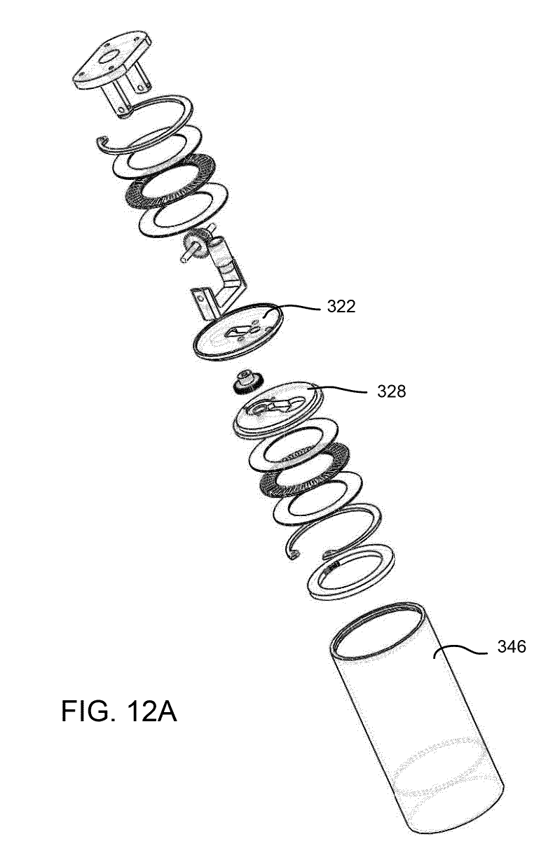

FIG. 12 shows a variation of the wrist device in FIG. 11 that functions similarly. It is labeled in accordance with corresponding elements of FIG. 11. This embodiment shows washers 348 placed next to thrust bearings 320 and 330 for smooth rotation of the wrist device inside outer sleeve 346. Wrist device 300 operates without an actuator housing as described in devices 10 and 200. The same function is obtained by containing the actuators with top motor plate 322 and bottom motor plate 328.

FIG. 12A shows a variation of the wrist device in FIG. 11 and the same device of FIG. 12 without the actuators. Actuation of the wrist device can be via standard motors or advanced smart material actuators placed along the wrist componentry or between top motor plate 322 and bottom motor plate 328 to elicit the wrist motions of flexion/extension and rotation.

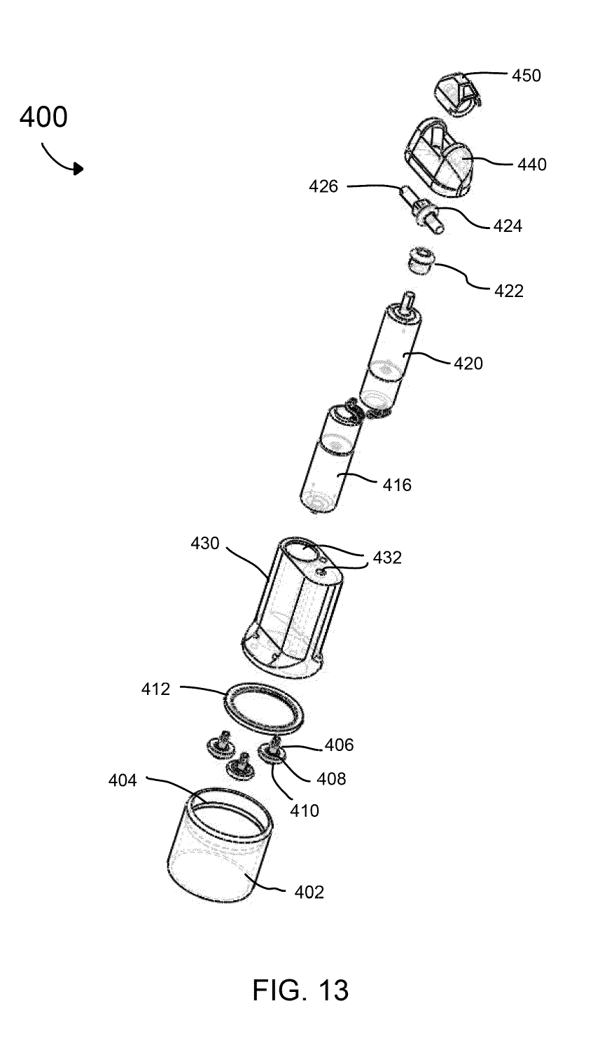

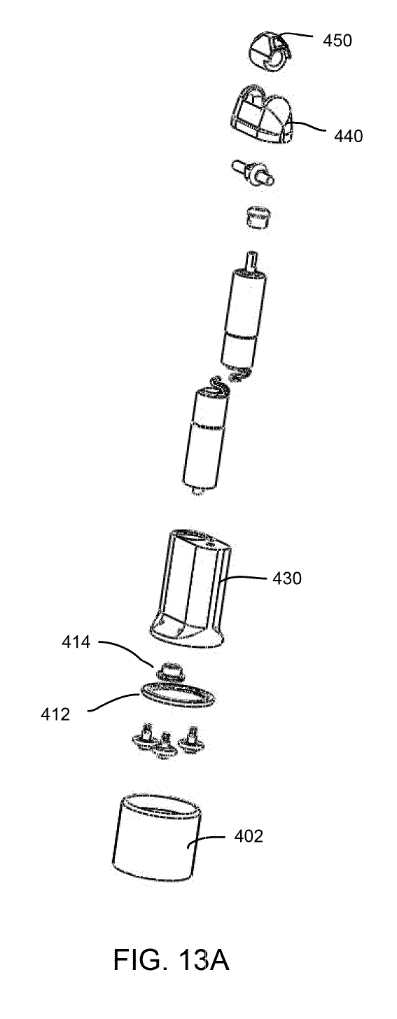

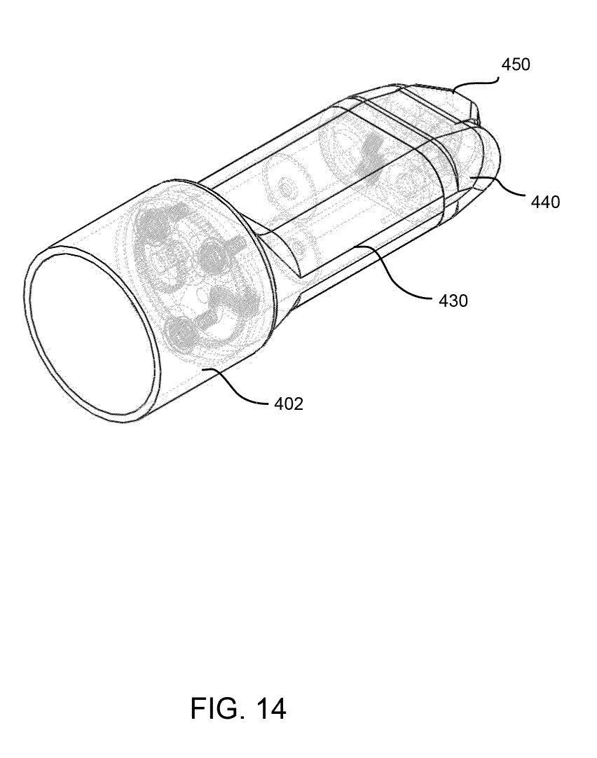

FIGS. 13, 13A, and 14 show a further embodiment of a wrist device. Wrist device 400 is designed to perform the same functions as wrist devices 10, 100, 200, and 300. Specific embodiments of the present invention include base sleeve 402, wrist housing 430, wrist cover 440, and flexor cover 450.

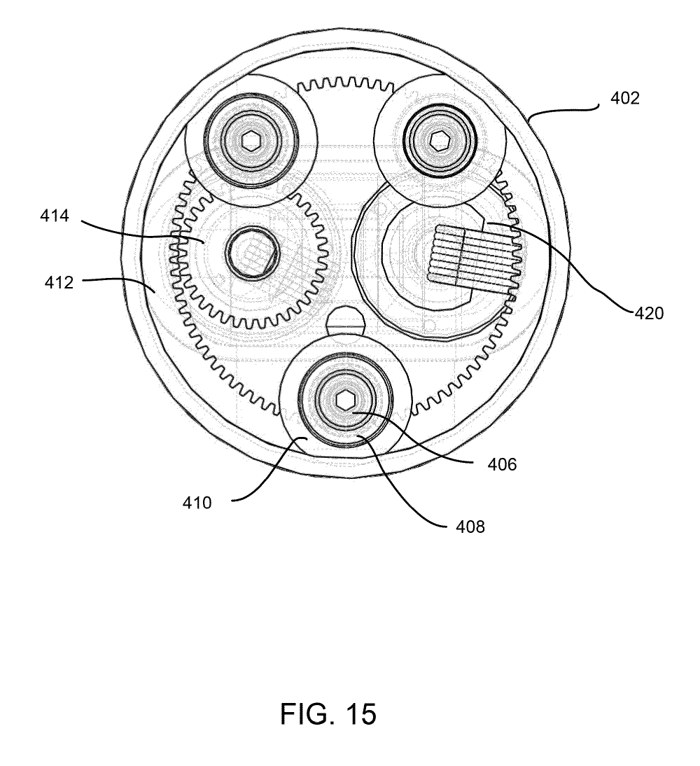

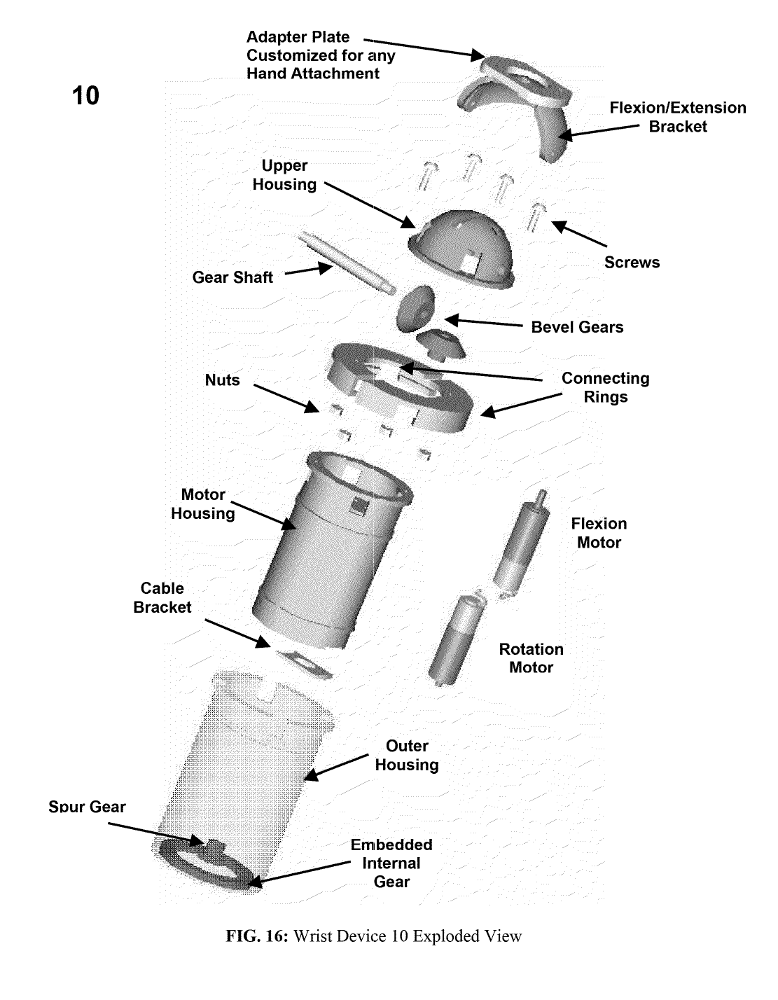

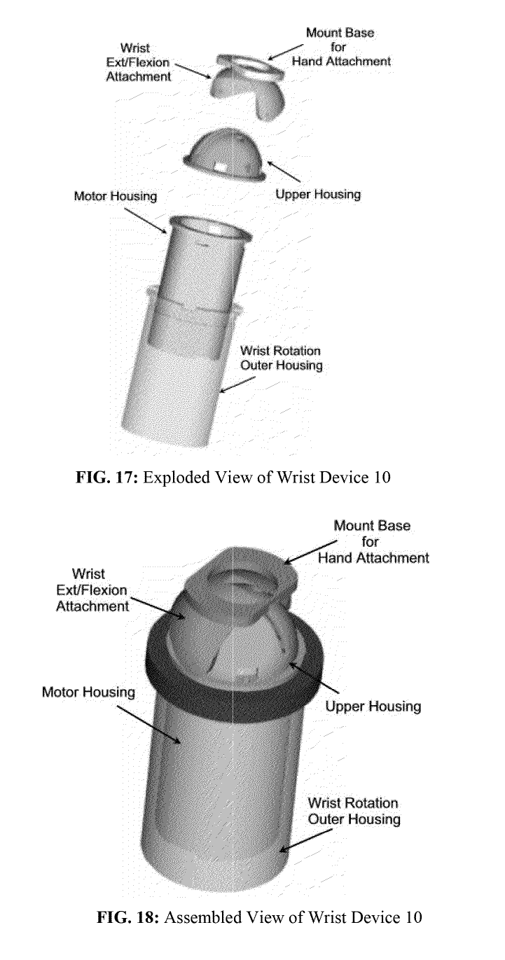

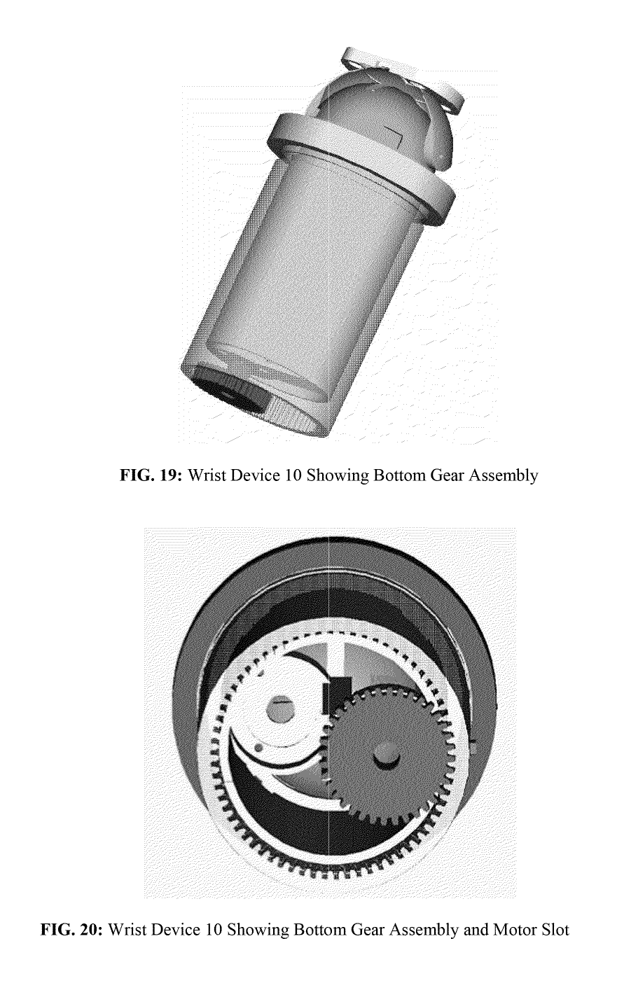

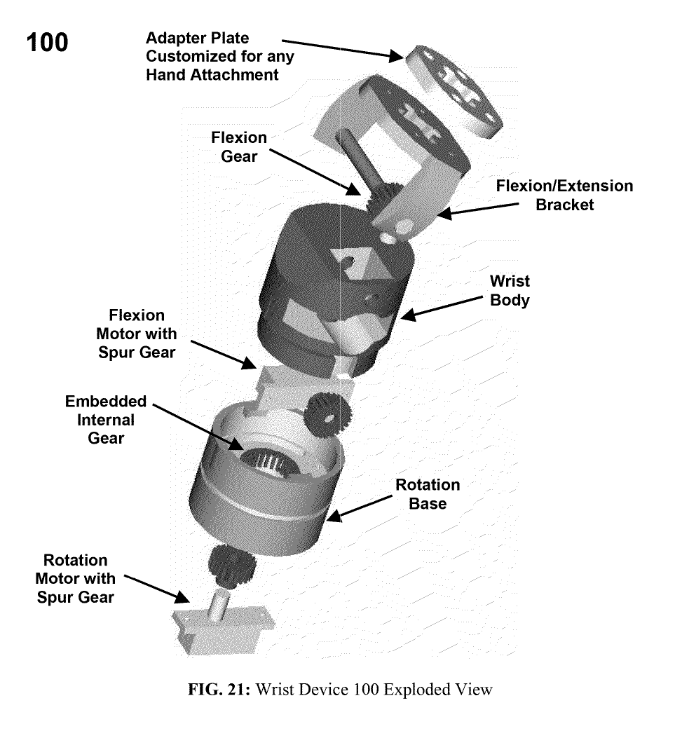

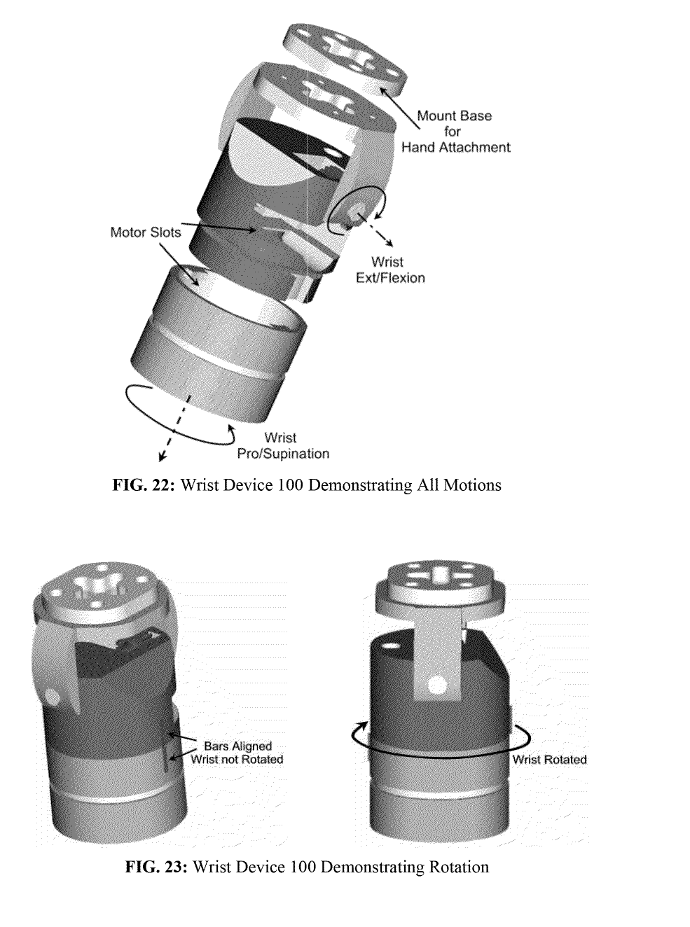

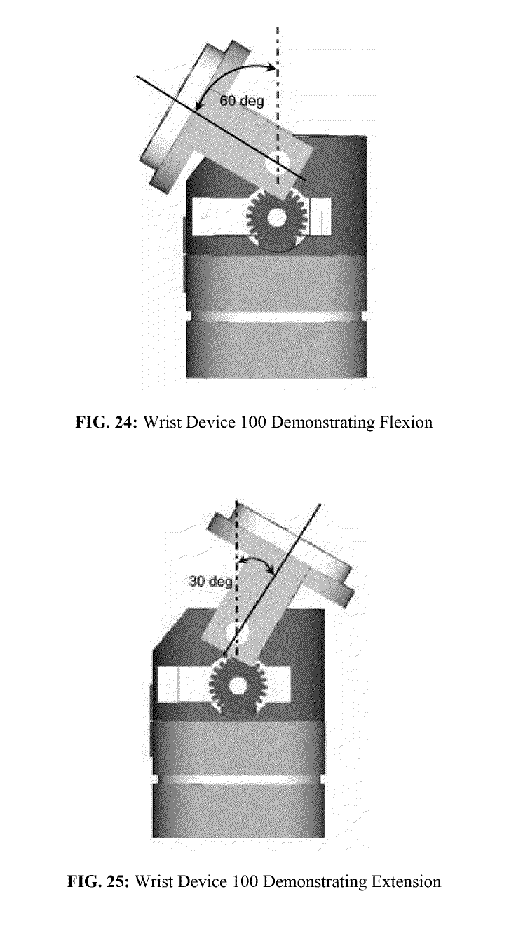

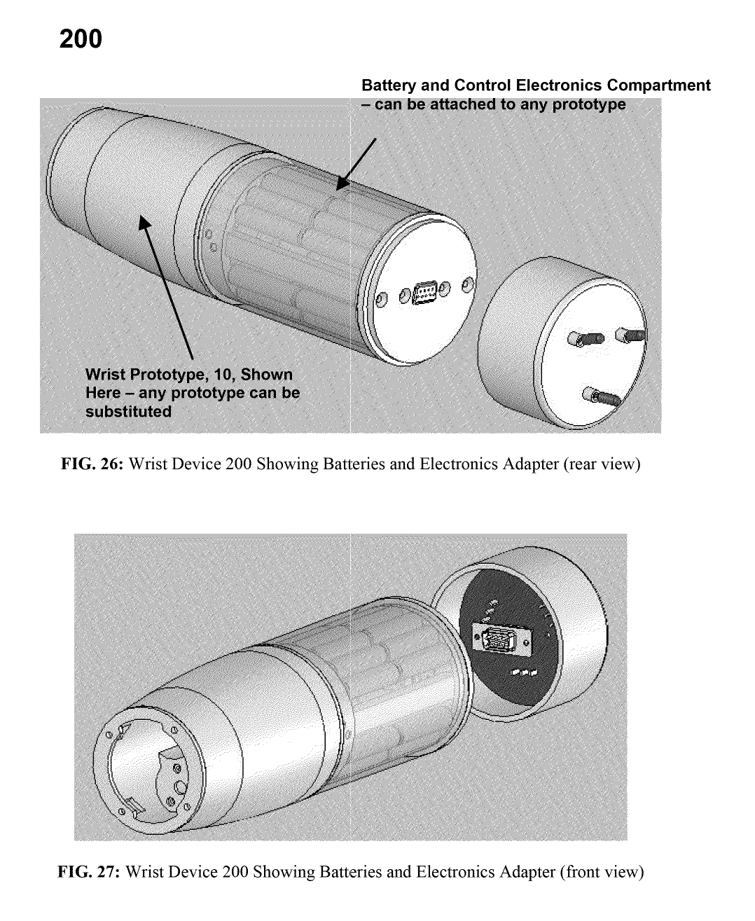

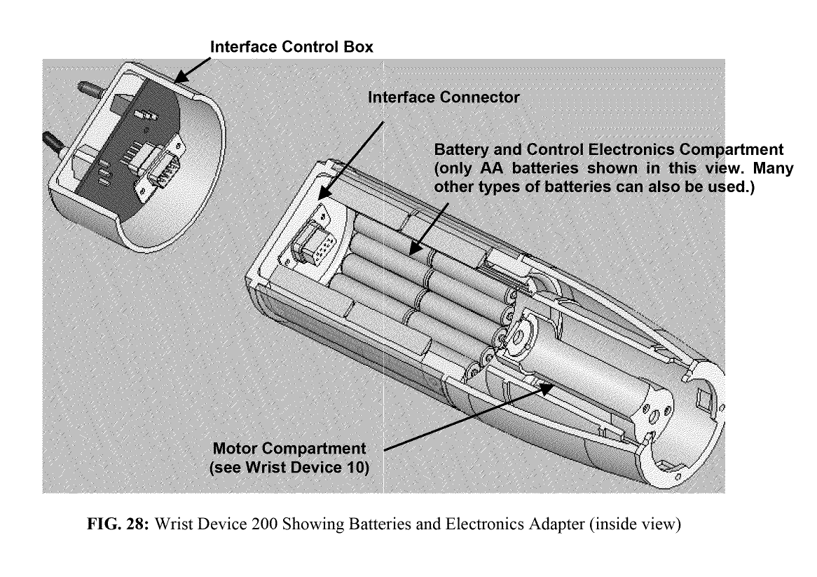

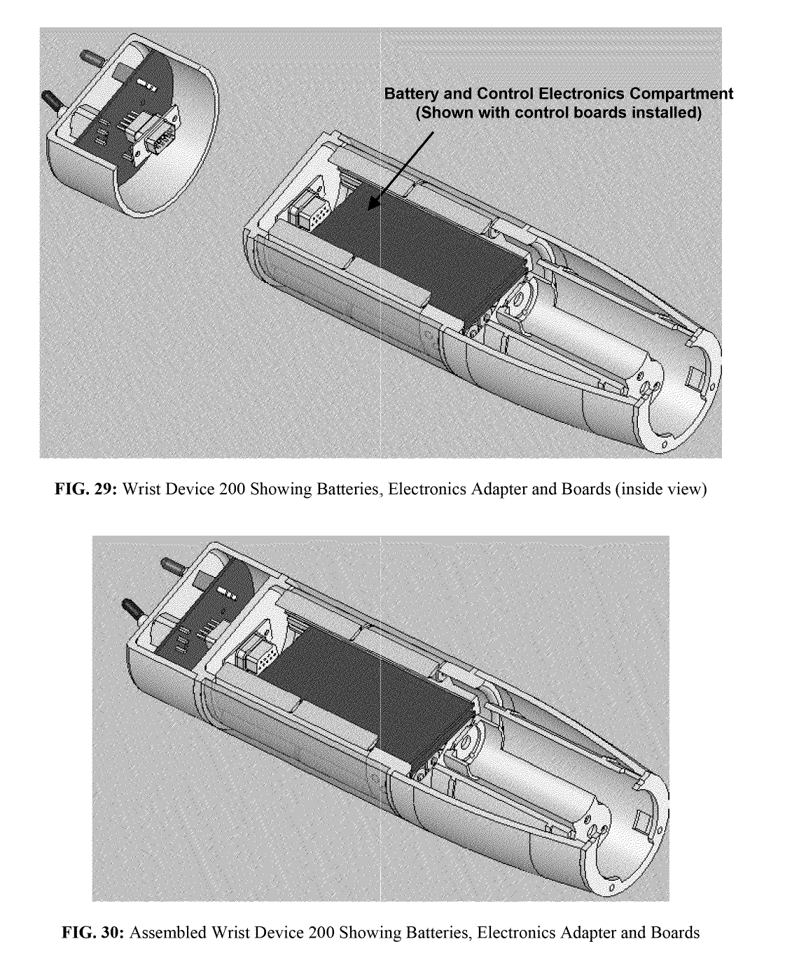

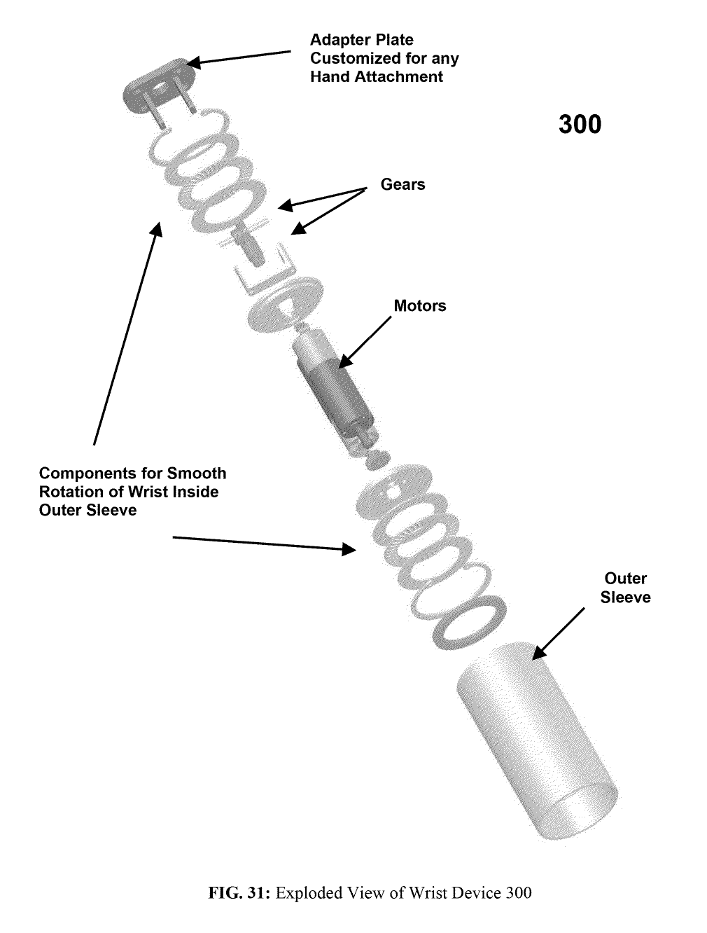

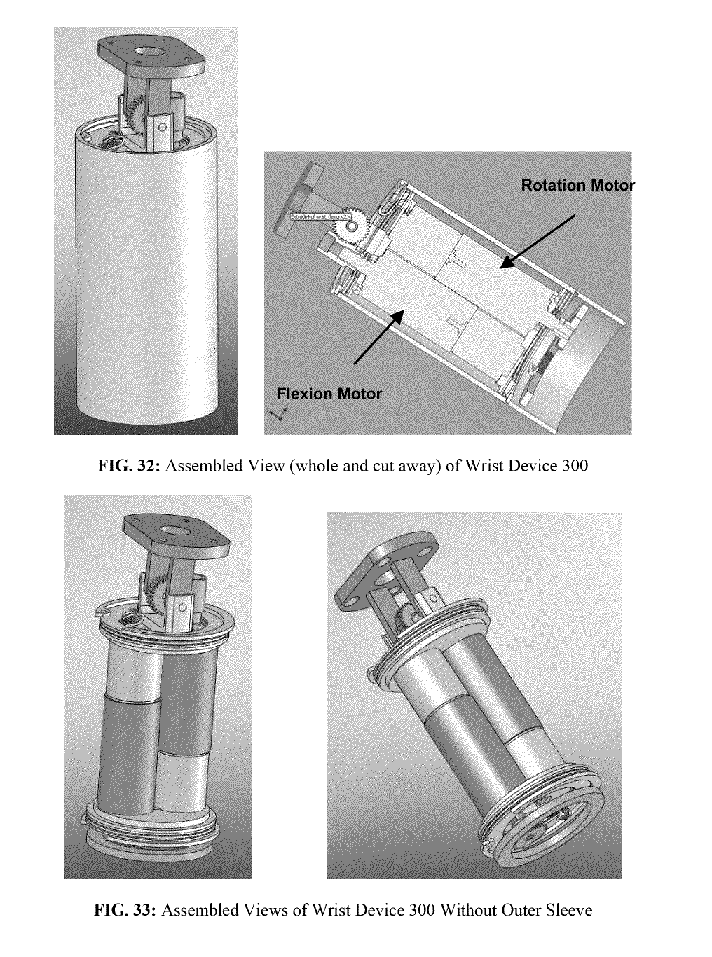

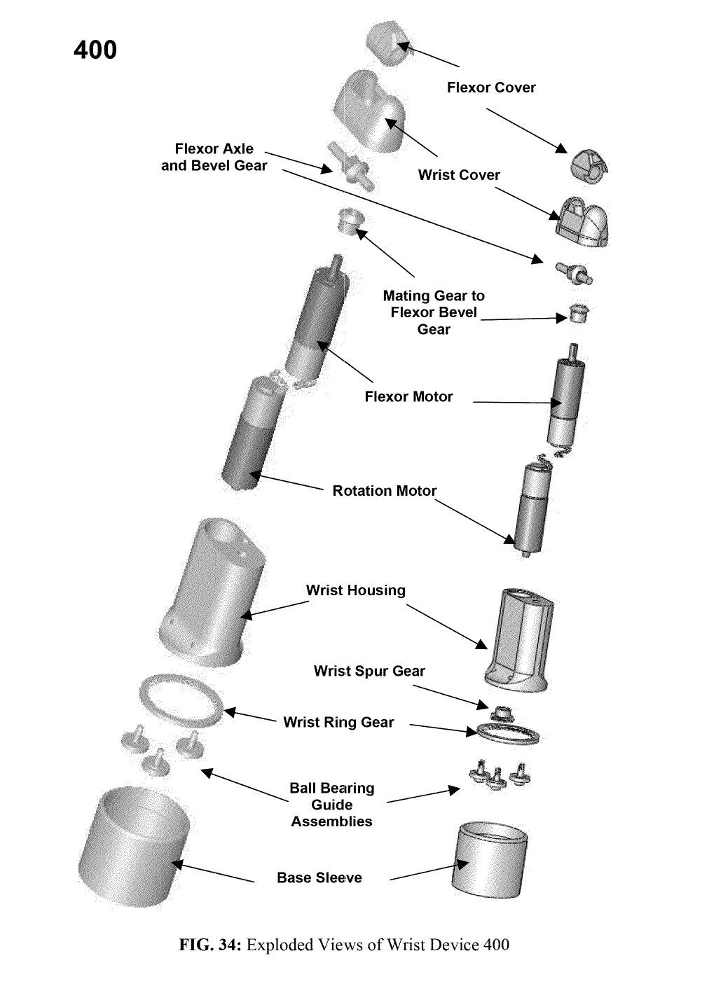

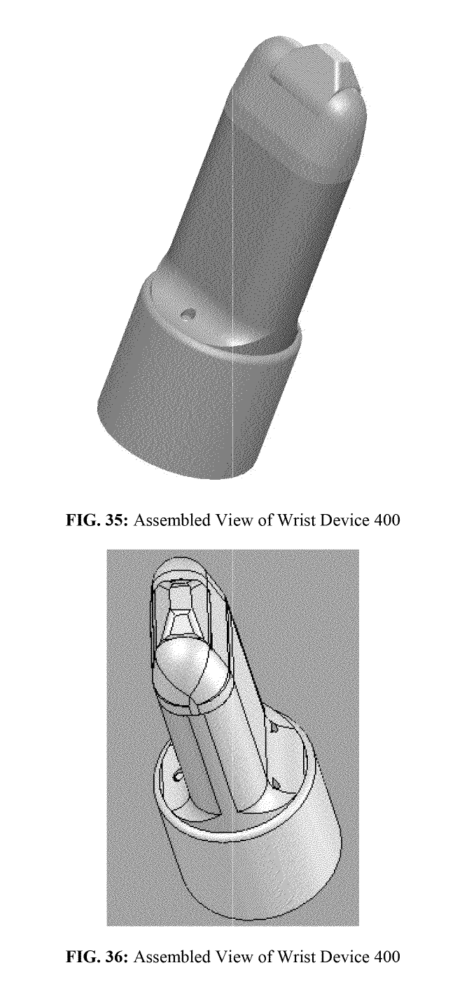

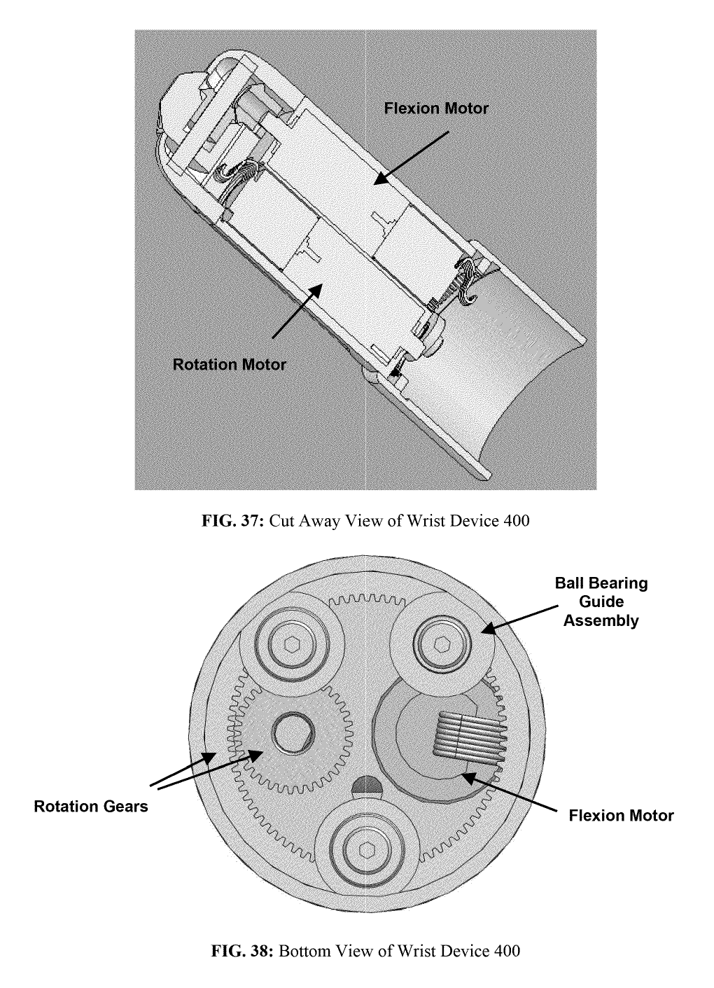

Base sleeve 402 serves as the anchor for the wrist rotation motion and is configured to be connected to a proximal prosthetic device and acts as a proximal mounting structure. Base sleeve 402 is hollow to receive the power and control components. Ball bearing guide assemblies comprised of a shoulder bolt 406, bearing 408, and roller 410 (see FIG. 15) hold wrist housing 430 to the base sleeve 402 securely while allowing wrist housing 430 to rotate. Wrist ring gear 412 is anchored to the base sleeve 402 in gear ring or slot 404. Wrist ring gear 412 is connected to rotating motor 416 and adapted to be attached to wrist drive gear 414 in order to rotate wrist housing 430 when rotating motor 416 is powered. Wrist housing 430 holds wrist rotating motor 416 and flexor motor 420. The motors are assembled and attached through holes or slots 432 in wrist housing 430. Bevel gear 422 attaches to flexor motor 420 and mates with flexor bevel gear 424, which is adapted to be attached to flexor axle 426. Bevel gear 422 and flexor bevel gear 424 transmit power from flexor motor 420 to flexor cover 450. Flexor axle 426 is adapted to be attached to flexor cover 450 through wrist cover 440 such that the flexor cover 450 flexes and extends when flexor motor 420 is powered. Flexor cover 450 is configured to be connected to a distal prosthetic terminal device.

Changes and modifications, additions and deletions may be made to the structures and methods recited above and shown in the drawings without departing from the scope or spirit of the invention and the following claims.

What is claimed is:

1. A powered two degree of freedom wrist unit adapted for use with an artificial limb, the wrist unit comprising:
a mounting platform adapted to connect to a modular terminal device;
the mounting platform connected to an articulated member operated by an actuator system allowing for externally powered flexion and extension of the wrist unit;
a rotation system associated with and operated by the actuator system allowing for externally powered rotation of the wrist unit;
command circuitry associated with the actuator system for receiving user generated inputs; and
a proximal mounting structure adapted to cooperate with and secure the wrist unit to a proximal prosthetic component.

2. The wrist unit of claim 1, wherein the user generated inputs comprise electromyographic signals, force resisting sensors, touchpads, linear transducers, pneumatic bladders, or switches.

3. The wrist unit of claim 1, wherein the actuator system comprises one or more actuators positioned to optimize rotational movement and flexion/extension movement.

4. The wrist unit of claim 4, wherein the actuators are inverted, or are placed in parallel or perpendicular with each other.

5. The wrist unit of claim 1, further comprising a lamination ring on the proximal mounting structure adapted to connect the wrist unit to a custom composite or thermoplastic socket.

6. The wrist unit of claim 1, wherein the proximal mounting structure is adapted to connect to a prosthetic elbow or bone anchored abutment.

7. The wrist unit of claim 1, wherein the proximal mounting structure comprises an end portion of an outer housing or a rotation base.

8. The wrist unit of claim 1, wherein the rotation system comprises an outer housing and an actuator housing, the actuator housing adapted to be received by and rotate within the outer housing and to house the actuator system.

9. The wrist unit of claim 1, wherein the rotation system comprises a rotation base and a wrist body, the wrist body adapted to be received by and rotate within the rotation base and to house the actuator system.

10. The wrist unit of claim 1, wherein the wrist unit provides increased mobility with ranges of at about 0-90° flexion, about 0-60° extension, and about 360° rotation.

11. The wrist unit of claim 1, wherein the wrist unit provides active lift of at about 1.5-10 ft-lbs and passive resist of about 10-30 ft-lbs.

12. The wrist unit of claim 1, wherein the actuator system comprises power transmission devices such as springs, or AC motors, DC motors, solenoids, linear and rotary motors, or smart material advanced actuators that are battery operated.

13. The wrist unit of claim 1, wherein the mounting platform, the articulated member, and the proximal mounting structure comprise aluminum, plastic, carbon fiber composite, or composites or alloys thereof.

14. The wrist unit of claim 1, wherein the command circuitry is adapted to be in electrical communication with user generated inputs, and wherein the command circuitry is further in electrical communication with the actuator system, such that when the user generated inputs are transmitted to the command circuitry, the actuator system responds by moving the wrist unit.

15. The wrist unit of claim 1, further comprising a modular terminal device comprising a hand, a hook, or activity-specific device attached to the mounting platform.

16. The wrist unit of claim 15, wherein the wrist unit is secured to a prosthetic component at the proximal mounting structure.

17. The wrist unit of claim 1, wherein the unit weighs about 0.5-1.00 pounds.

18. A powered prosthetic unit adapted to provide powered flexion, extension, and rotation to a modular artificial limb.

19. The powered prosthetic unit of claim 18, wherein the unit is battery powered.
